(19) United States
(12) Patent Application Publication
(10) Pub. No.: US 2026/0009054 A1
(43) Pub. Date: Jan. 8, 2026

(54) METHOD TO PRODUCE THE ANTI-MICROBIAL DITERPENOID LEUBETHANOL AND RELATED SERRULATANE-TYPE DITERPENES

(71) Applicant: BOARD OF TRUSTEES OF MICHIGAN STATE UNIVERSITY, East Lansing, MI (US)

(72) Inventors: Garret Miller, Lansing, MI (US); Wajid Waheed Bhat, Haslet, MI (US); Emily R. Lanier, Haslett, MI (US); Sean Johnson, Bedford, MA (US); Björn Hamberger, OKEMOS, MI (US); Davis T. Mathieu, EAST LANSING, MI (US)

(21) Appl. No.: 19/252,451

(22) Filed: Jun. 27, 2025

Related U.S. Application Data
(62) Division of application No. 17/905,749, filed on Sep. 6, 2022, now Pat. No. 12,378,579.
(60) Provisional application No. 62/986,286, filed on Mar. 6, 2020.

(51) Int. Cl. C12P 7/22

(52) U.S. Cl. CPC C12P 7/22; C12Y 114/14; C12Y 205/01087; C12Y 402/03112

(57) ABSTRACT

The disclosure relates to a method of making at least one serrulatane comprising contacting a terpene or a terpenoid substrate with at least one of a cis-prenyl transferase, a terpene synthase, and a cytochrome P450. The disclosure also relates to an expression system comprising one or more expression cassettes, each expression cassette comprising a promoter operably linked to a nucleic acid segment encoding at least one of: a cis-prenyl transferase, a terpene synthase, and a cytochrome P450. The disclosure also relates to a host cell comprising an expression system comprising one or more expression cassettes, each expression cassette comprising a promoter operably linked to a nucleic acid segment encoding at least one of: a cis-prenyl transferase, a terpene synthase, and a cytochrome P450.

### CROSS-REFERENCE TO RELATED APPLICATIONS

This application is a divisional application of U.S. Ser. No. 17/905,749, filed Sep. 6, 2022, which is a U.S. National Stage Filing under 35 U.S.C. 371 of International Patent Application Serial No. PCT/US2021/021156, filed Mar. 5, 2021, and published as WO 2021/178850 A1 on Sep. 10, 2021, which claims the benefit of priority from U.S. Provisional Appl. Ser. No. 62/986,286, filed Mar. 6, 2020, all of which are incorporated by reference as if fully set forth herein.

### STATEMENT OF GOVERNMENT SUPPORT

This invention was made with government support under GM110523 awarded by the National Institutes of Health. The government has certain rights in the invention.

### INCORPORATION BY REFERENCE OF SEQUENCE LISTING

The sequence listing contained in the file named “P14833US02.xml,” which is 12,461 bytes (measured in MS-Windows®) and was created on Jun. 26, 2025, is incorporated herein by reference in its entirety.

### BACKGROUND

Plant diterpenes occupy a unique molecular space with critical pharmaceutical applications over a diverse spectrum including anti-cancer, anti-microbial and immunomodulatory properties. In addition, plant-derived terpenoids have a wide range of commercial and industrial uses. Examples of uses for terpenoids include specialty fuels, agrochemicals, fragrances, nutraceuticals and pharmaceuticals. However, currently available methods for synthesis, extraction, and purification of terpenoids from the native plant sources have limited economic sustainability. Moreover, currently available methods for do not provide the substrates and methods for biosynthesis of non-natural terpenoids.

Cost-effective synthesis and access to analogs of plant diterpenoids and their derivatives is technologically limited on the levels of isolation, purification, detection, and synthesis.

### SUMMARY

Described herein is a pathway for manufacturing serrulatanes that are useful as therapeutic agents. The pathway includes use of the cytochrome P450 enzyme such as CYP71D616, which can catalyze the formation of the anti-tuberculosis compound leubethanol. The biosynthetic enzymes can, for example, be from the plant Leucophyllum frutescens.

### DESCRIPTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

New terpene biosynthetic methods for making new types of terpenes are described herein. Diterpenes occupy a unique molecular space with critical pharmaceutical applications over a diverse spectrum including anti-microbial, anti-cancer, immunomodulatory and psychoactive properties. Many diterpenoids are currently recognized as “drugs” (351 of over 12,500 are listed in the Dictionary of Natural Products, Taylor and Francis Group, DNP 28.1). A key challenge, however, is optimization of these compounds, and derivatization is usually not synthetically tractable.

Serrulatane diterpenoids are natural products found in plants from multiple genera within the figwort family (Scrophulariaceae). Many of these compounds have antimicrobial properties and they share a common diterpene backbone. One example, leubethanol from Texas sage, Leucophyllum frutescens, has demonstrated activity against multi-drug resistant tuberculosis. The structure for leubethanol (1) is shown below.

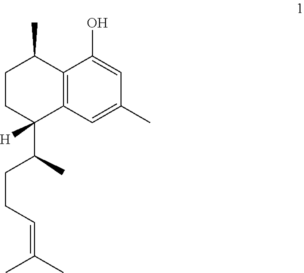 [SMILES: CC(C)=CCC[C@H](C)[C@@H]1CC[C@@H](C)c2c(O)cc(C)cc21]

Despite potential therapeutic relevance, the biosynthesis of serrulatane diterpenoids has not been previously reported. Access to these molecules is currently limited to total chemical synthesis or extraction from natural sources.

Described herein is the full biosynthetic pathway to serrulatane diterpenoids. A short-chain cis-prenyl transferase (LfCPT1) first produces the rare diterpene precursor nerylneryl diphosphate, which is cyclized by an unusual plastidial terpene synthase (LfTPS1) into the characteristic serrulatane diterpene backbone. Final conversion to leubethanol is catalyzed by a cytochrome P450 (CYP71D616) of the CYP71 clan. This pathway documents the first case of a short-chain cis-prenyl transferase in the Lamiales order of plants and provides methods for biosynthesis of diverse diterpenoids in Eremophila. LfTPS1 represents an example of neofunctionalization and acceptance of a novel substrate after localization to the plastid. Biosynthetic access to the serrulatane backbone and leubethanol provides a pathway for manufacture of complex serrulatane diterpenoids, a diverse class of promising antimicrobial therapeutics.

Examples of serrulatanes that can be synthesized using the methods described herein include compounds of the general formula:

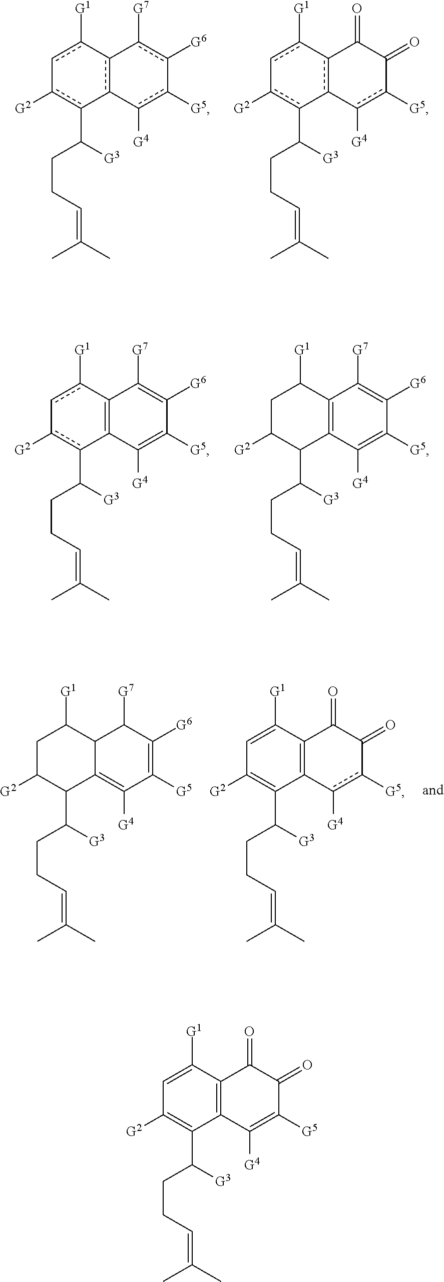

wherein:
G¹ is substituted or unsubstituted alkyl (e.g., (C₁-C₅)-alkyl, such as methyl);
G² is H, substituted or unsubstituted alkyl (e.g., (C₁-C₅)-alkyl, such as methyl), or OG⁸, wherein G⁸ is H, substituted or unsubstituted alkyl (e.g., (C₁-C₅)-alkyl), or acyl (e.g., (C₁-C₅)-alkyl-C(O)—, such as acetyl);
G³ is substituted or unsubstituted alkyl (e.g., (C₁-C₅)-alkyl, such as methyl);
G⁴ is H, substituted or unsubstituted alkyl (e.g., (C₁-C₅)-alkyl), or G⁴ and G³, together with the atoms to which they are attached, can form a five- or six-membered heterocyclyl;
G⁵ is substituted or unsubstituted alkyl (e.g., (C₁-C₅)-alkyl, such as methyl) or C(O)OG⁸ (e.g., CO₂H and esters); and
G⁶ and G⁷ are each, independently, H or OG⁸ (e.g., where G⁸ is (C₁-C₅)-alkyl-C(O)—, such as acetyl), such as the following compounds:

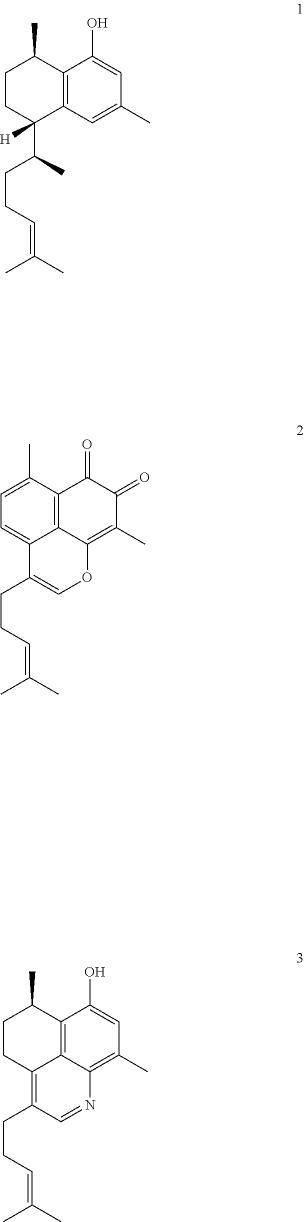 [SMILES: CC(C)=CCC[C@H](C)[C@@H]1CC[C@@H](C)c2c(O)cc(C)cc21 ; CC(C)=CCCC1=COC2=C(C)C(=O)C(=O)c3c(C)ccc1c32 ; CC(C)=CCCc1cnc2c(C)cc(O)c3c2c1CC[C@H]3C]

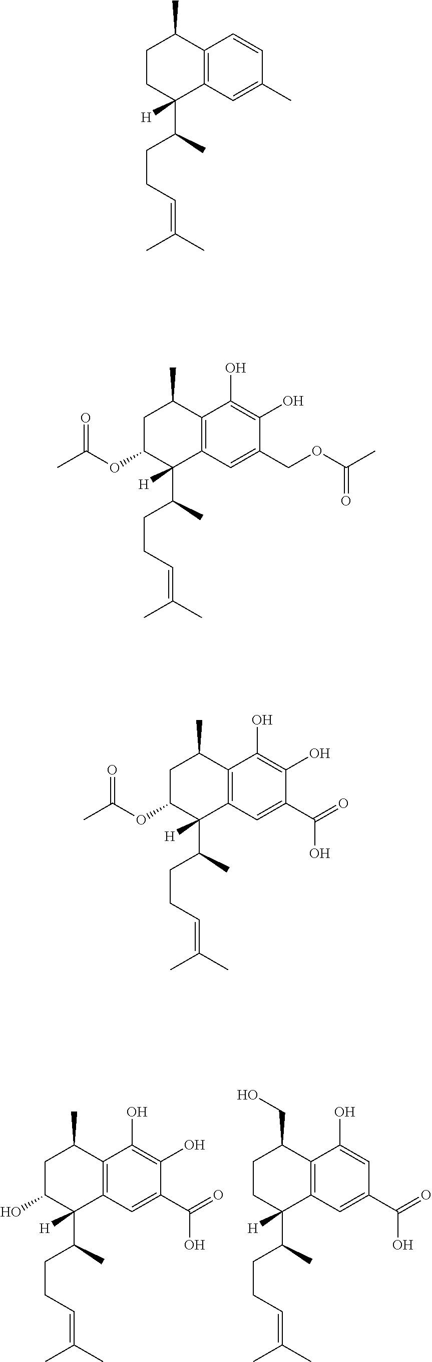

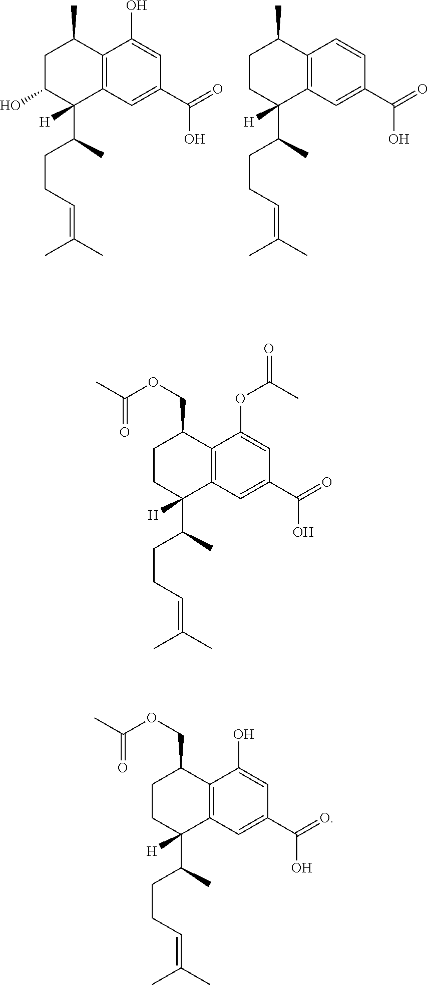

As demonstrated herein, the pathway for serrulatane diterpenoids proceeds via an uncommon diterpene precursor which is then cyclized into a diterpene backbone. Oxidative functionalization yielded the diterpenoid structure shared across all serrulatanes. Three enzymes constitute the full biosynthetic pathway to leubethanol in L. frutescens. While the vast majority of diterpenoids originate from GGPP, the serrulatane diterpenoid pathway involves a short-chain cis-prenyl transferase (cis-PT) which produces the uncommon diterpene precursor (Z,Z,Z)-nerylneryldiphosphate (NNPP—the all-cis stereoisomer of GGPP). This is then cyclized to the shared serrulatane backbone by a terpene synthase (TPS), which exclusively uses NNPP as a substrate, and which is a member of the primarily-sesquiterpene (C15) synthase TPS-a subfamily. Finally, this serrulatane backbone is converted to leubethanol by a cytochrome P450 of the diverse CYP71 clan that harbors many P450s of terpene specialized metabolism. The full pathway to the serrulatane leubethanol is illustrated herein within the heterologous Nicotiana benthamiana system.

Hence, the serrulatane synthetic route can involve use of cis-prenyl transferase to generate the diterpene precursor (Z,Z,Z)-nerylneryldiphosphate (NNPP), for example from substrates dimethylallyl pyrophosphate (DMAPP) and isopentenyl pyrophosphate (IPP). The structure of (Z,Z,Z)-nerylneryldiphosphate (NNPP) is shown below.

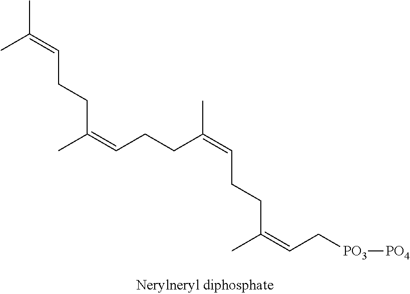

An example of a sequence for a cis-prenyl transferase from Leucophyllum frutescens (LfCPT1) that can synthesize this reaction is provided as SEQ ID NO:1.

A nucleotide sequence for the Leucophyllum frutescens LfCPT1 with SEQ ID NO: 1 is provided as SEQ ID NO:2.

The (Z,Z,Z)-nerylneryldiphosphate (NNPP) is then cyclized to the shared serrulatane backbone by a terpene synthase (TPS) to provide the following compound.

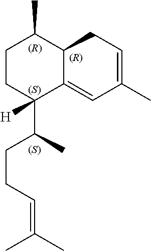

Compounds such as the compound of formula 1 can then be accessed as follows:

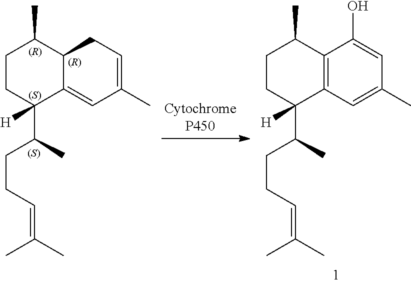

An example of a sequence for a terpene synthase from Leucophyllum frutescens (LfTPS1) that can synthesize this cyclization reaction is provided as SEQ ID NO:3.

A nucleotide sequence for the Leucophyllum frutescens LfTPS1 with SEQ ID NO: 3 is provided as SEQ ID NO:4.

An example of a sequence of a cytochrome P450 (CYP71D616) enzyme that can convert this cyclized serrulatane backbone to leubethanol is provided as SEQ ID NO:5.

A nucleotide sequence for the Leucophyllum frutescens CYP71D616 with SEQ ID NO:5 is provided as SEQ ID NO:6.

Therefore, described herein is a chemical strategy to synthesize diterpene class.

Enzymatic biosynthesis of pharmaceutically active compounds is increasingly important for securing access to relevant chemistries, scalability of production, and long-term reduction in cost for synthesis of serrulatanes. Genetic information was used to reconstruct the pathways to serrulatanes, especially the pharmacologically active serrulatanes.

The enzymes described herein can have some sequence variations. For example, enzymes described herein can have one or more deletions, insertions, replacements, or substitutions in a part of the enzyme. The enzyme(s) described herein can have, for example, at least 60%, or at least 70%, or at least 80%, or at least 90%, or at least 93%, or at least 95%, or at least 96%, or at least 97%, or at least 98%, or at least 99% sequence identity to a sequence described herein.

In some cases, enzymes can have conservative changes such as one or more deletions, insertions, replacements, or substitutions that have no significant effect on the activities of the enzymes. Examples of conservative substitutions are provided below in Table 1A.

TABLE 1A
Conservative Substitutions

| Type of Amino Acid | Substitutable Amino Acids |
|---|---|
| Hydrophilic | Ala, Pro, Gly, Glu, Asp, Gln, Asn, Ser, Thr |
| Sulfhydryl | Cys |
| Aliphatic | Val, Ile, Leu, Met |
| Basic | Lys, Arg, His |
| Aromatic | Phe, Tyr, Trp |

A variety of additional enzymes can be used in the methods described herein. For example, the methods can also include use of one or more transcription factor, cis-prenyl transferase, terpene synthase, cytochrome P450, cytochrome P450 reductase, 1-deoxy-D-xylulose 5-phosphate synthase (DXS), 1-deoxy-D-xylulose 5-phosphate-reducto-isomerase, cytidine 5′-diphosphate-methylerythritol (CDP-ME) synthetase (IspD), 2-C-methyl-d-erythritol 2,4-cyclodiphosphate synthase (IspF), geranylgeranyl diphosphate synthase (GGDPS), HMG-COA synthase, HMG-COA reductase (HMGR), mevalonic acid kinase (MVK), phosphomevalonate kinase (PMK), mevalonate-5-diphosphate decarboxylase (MPD), isopentenyl diphosphate isomerase (IDI), abietadiene synthase (ABS), farnesylpyrophosphate synthase (FPPS), ribulose bisphosphate carboxylase, squalene synthase (SQS), patchoulol synthase, or WRI1 protein.

Such enzymes can be obtained from organisms such as Leucophyllum frutescens (Lf), Tripterygium wilfordii (Tw), Euphorbia peplus (Ep), Coleus forskohlii (Cf), Ajuga reptans (Ar), Perovskia atriciplifolia (Pa), Nepeta mussini (Nm), Origanum majorana (Om), Hyptis suaveolens (Hs), Grindelia robusta (Gr), Leonotis leonurus (LI), Marrubium vulgare (Mv), Vitex agnus-castus (Vac), Euphorbia peplus (Ep), Ricinus communis (Rc), Daphne genkwa (Dg), Zea mays (Zm), and other organisms. Co-pending U.S. Provisional Application Ser. No. 62/930,898, filed Nov. 5, 2019 provides further information on these enzymes. U.S. Provisional Application Ser. No. 62/930,898, filed Nov. 5, 2019, is incorporated herein by reference in its entirety.

### Substrates

The methods described herein can include use of different substrates to produce a variety of different products.

Taking advantage of natural substrate promiscuity, precursor-directed biosynthesis was used to generate variants of the drugs in the family of non-ribosomal peptides, polyketides and non-natural indole alkaloids. Modification of natural products can provide analogs with improved or novel medicinal properties. To that end, the disclosure relates to substrates of the formula (I), (Ia) or (II):

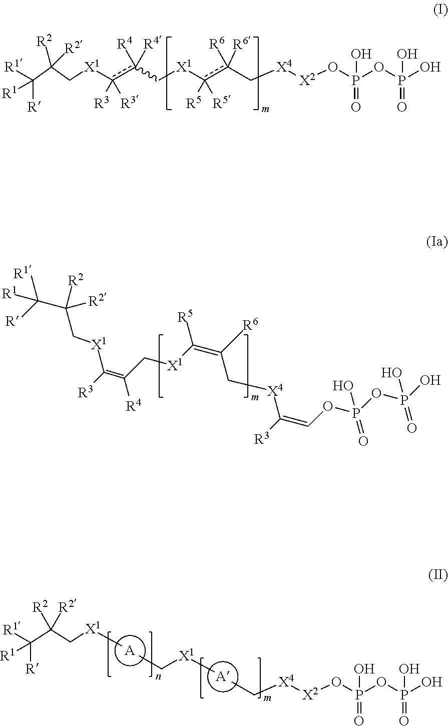

wherein:
m is an integer from 0 to 3 (e.g., 1 or 2), with the understanding that if m is 2 or 3, each repeating subunit can be the same or different;
n is an integer from 0 to 1;
the dashed lines (-----) represent a double bond when R^{3′} and R^{4′} are absent or
when R^{5′} and R^{6′} are absent,
A and A′ are each independently cycloalkyl, aryl or heterocyclyl, each of which can be optionally substituted;
X¹ is a heteroatom, -X³-alkyl, -alkyl-X³- or alkyl, wherein X³ is a heteroatom or alkyl or X¹ is:

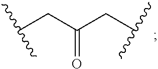 [SMILES: *CC(=O)C*]

R¹ and R² form a double bond or an epoxide;
each R′, R^{1′}, R², R^{2′} and R³-R⁶ is, independently, H, alkyl, halo, aryl, and alkylaryl;
R^{3′} and R^{4′} are absent or R^{3′} and R^{4′}, together with the carbon atoms to which they are attached, form an epoxide, a cycloalkyl group, an aryl group or a heterocyclyl group;
R^{5′} and R^{6′} are absent or R^{5′} and R^{6′}, together with the carbon atoms to which they are attached, form an epoxide, a cycloalkyl group, an aryl group or a heterocyclyl group;
X² is a bond, alkenyl or acyl; and
X⁴ is a absent, a heteroatom or alkyl;
with the proviso that the compound of the formula (I) is not a compound of the formula:

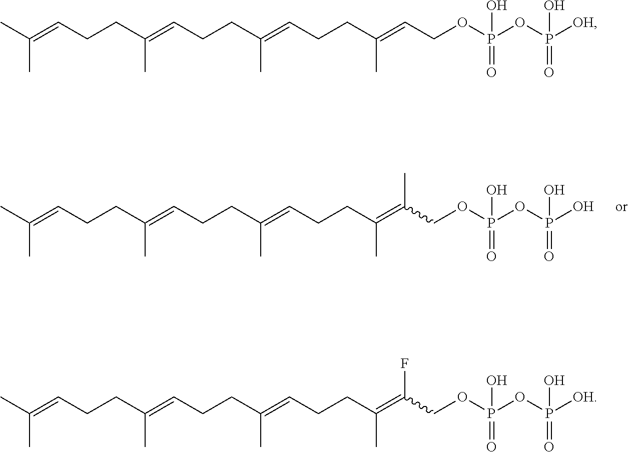 [SMILES: CC(C)=CCC/C(C)=C/CC/C(C)=C/CC/C(C)=C/COP(=O)(O)OP(=O)(O)O ; CC(C)=CCC/C(C)=C/CC/C(C)=C/CCC(C)=C(C)COP(=O)(O)OP(=O)(O)O ; CC(C)=CCC/C(C)=C/CC/C(C)=C/CCC(C)=C(F)COP(=O)(O)OP(=O)(O)O]

Examples of compounds of the formula (I) include compounds of the formula:

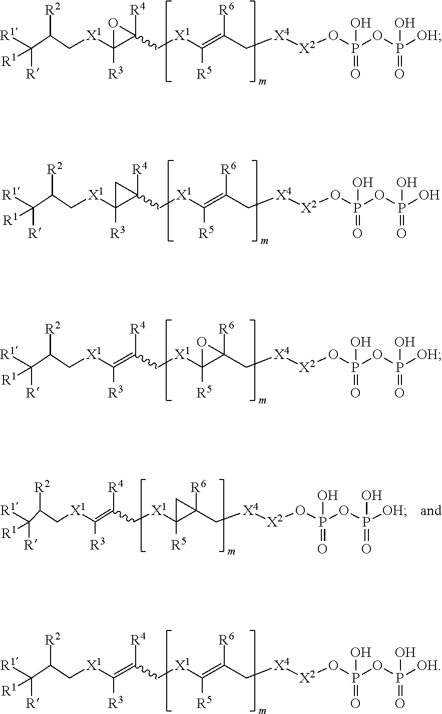

Examples of the formula (II) include compounds of the formula:

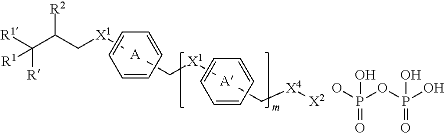

Examples of compounds of the formula (I) include compounds wherein if X¹ is a heteroatom, the heteroatom is oxygen. Other examples of compounds of the formula (I) include compounds wherein X³ is oxygen or C₁-C₅-alkyl, such as —CH₂— and C₂-C₃-alkyl. Still other examples of compounds of the formula (I) include compounds wherein R₃-R₆ are each H or C₁-C₅-alkyl, such as methyl and C₂-C₃-alkyl. Still other examples of compounds of the formula (I) include compounds wherein R³ and R⁵ are each H or C₁-C₅-alkyl, such as methyl and C₂-C₃-alkyl; and R⁴ and R⁶ are each H. Yet other examples of compounds of the formula (I) include compounds wherein m is 1 or 2. In other examples, m is 0. Other examples of compound of the formula (I) include compounds wherein X² is an alkenyl group of the formula:

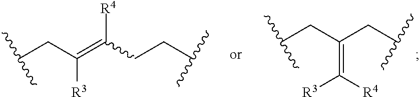

or an acyl group of the formula:

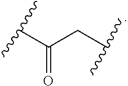 [SMILES: *CC(*)=O]

Examples of compounds of the formula (I) include compounds of the formulae:

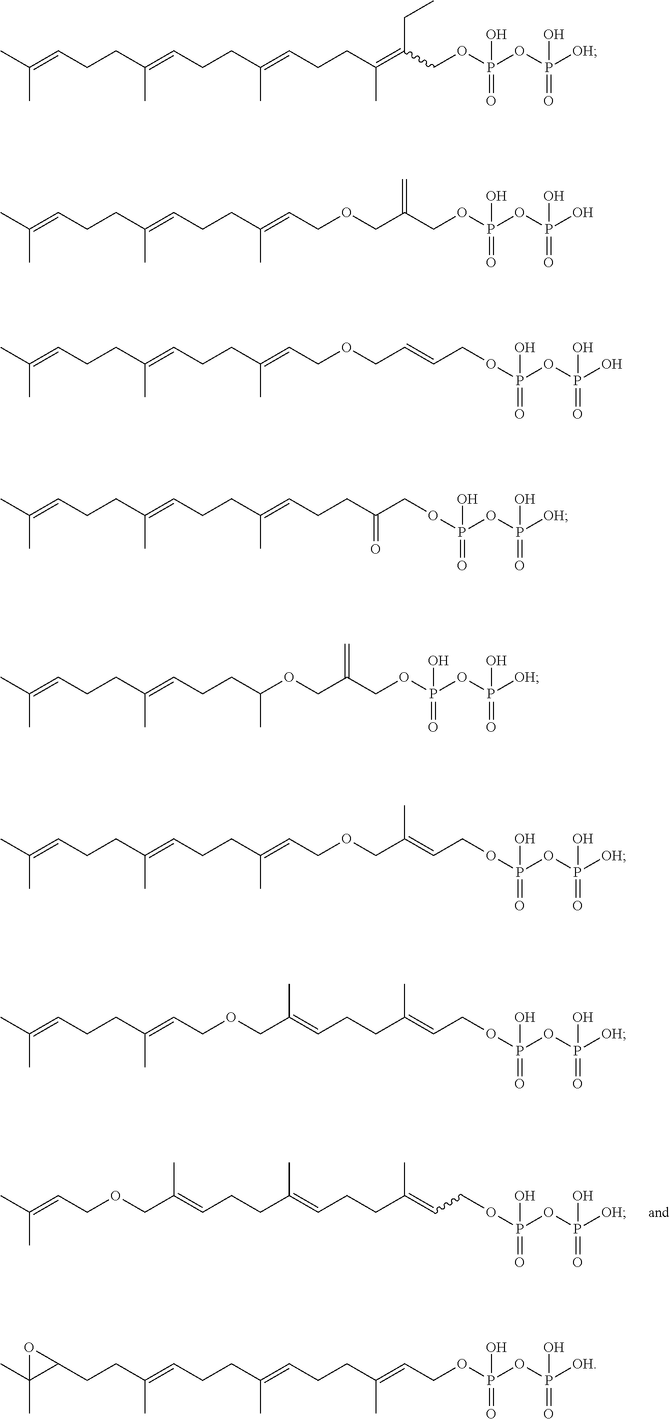

The compounds of the formula (I) or (II) can be enzymatically transformed into terpenoids having compound cores of the formula:

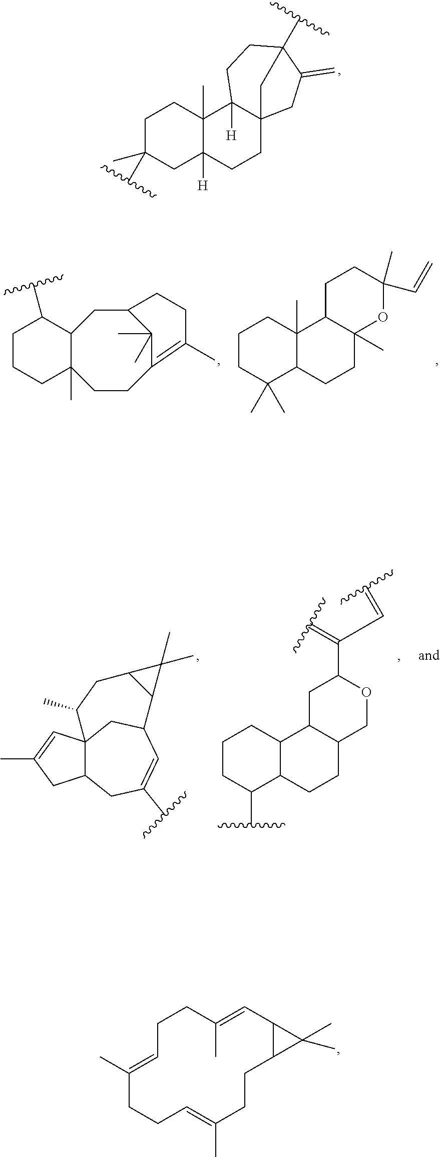

which correspond to the cores of stevioside, Taxol®, Forskolin, Picato®, and Salvinorin, Casbene, CPP respectively; or the core shared by CPP, LPP, PgPP, and KPP, namely:

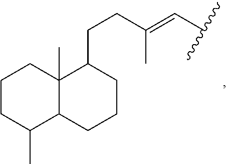 [SMILES: */C=C(\C)CCC1CCCC2C(C)CCCC12C]

and derivatives thereof, wherein derivatives can comprise additional double bonds, alkyl groups, hydroxy groups, acyl groups, and the like, dispersed about the cores.

As used herein, the term “heteroatom” refers to heteroatom such as, but not limited to, NR⁷, O, and SOₓ, wherein R⁷ is H, alkyl or arylalkyl, and x is 0, 1 or 2.

The term “alkyl” as used herein refers to substituted or unsubstituted straight chain, branched and cyclic, saturated mono-or bi-valent groups having from 1 to 20 carbon atoms, 10 to 20 carbon atoms, 12 to 18 carbon atoms, 6 to about 10 carbon atoms, 1 to 10 carbons atoms, 1 to 8 carbon atoms, 2 to 8 carbon atoms, 3 to 8 carbon atoms, 4 to 8 carbon atoms, 5 to 8 carbon atoms, 1 to 6 carbon atoms, 2 to 6 carbon atoms, 3 to 6 carbon atoms, or 1 to 3 carbon atoms. Examples of straight chain mono-valent (C₁-C₂₀)-alkyl groups include those with from 1 to 8 carbon atoms such as methyl (i.e., CH₃), ethyl, n-propyl, n-butyl, n-pentyl, n-hexyl, n-heptyl, n-octyl groups. Examples of branched mono-valent (C₁-C₂₀)-alkyl groups include isopropyl, iso-butyl, sec-butyl, t-butyl, neopentyl, and isopentyl. Examples of straight chain bi-valent (C₁-C₂₀)alkyl groups include those with from 1 to 6 carbon atoms such as —CH₂—, —CH₂CH₂—, —CH₂CH₂CH₂—, —CH₂CH₂CH₂CH₂—, and —CH₂CH₂CH₂CH₂CH₂—. Examples of branched bi-valent alkyl groups include —CH(CH₃)CH₂— and —CH₂CH(CH₃)CH₂—. Examples of cyclic alkyl groups include cyclopropyl, cyclobutyl, cyclopentyl, cyclohexyl, cyclooctyl, bicyclo[1.1.1]pentyl, bicyclo[2.1.1]hexyl, and bicyclo[2.2.1]heptyl. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. In some embodiments, alkyl includes a combination of substituted and unsubstituted alkyl. As an example, alkyl, and also (C₁)alkyl, includes methyl and substituted methyl. As a particular example, (C₁)alkyl includes benzyl. As a further example, alkyl can include methyl and substituted (C₂-C₈)alkyl. Alkyl can also include substituted methyl and unsubstituted (C₂-C₈)alkyl. In some embodiments, alkyl can be methyl and C₂-C₈ linear alkyl. In some embodiments, alkyl can be methyl and C₂-C₈ branched alkyl. The term methyl is understood to be —CH₃, which is not substituted. The term methylene is understood to be —CH₂—, which is not substituted. For comparison, the term (C₁)alkyl is understood to be a substituted or an unsubstituted —CH₃ or a substituted or an unsubstituted —CH₂—. Representative substituted alkyl groups can be substituted one or more times with any of the groups listed herein, for example, cycloalkyl, heterocyclyl, aryl, amino, haloalkyl, hydroxy, cyano, carboxy, nitro, thio, alkoxy, and halogen groups. As further example, representative substituted alkyl groups can be substituted one or more fluoro, chloro, bromo, iodo, amino, amido, alkyl, alkoxy, alkylamido, alkenyl, alkynyl, alkoxycarbonyl, acyl, formyl, arylcarbonyl, aryloxycarbonyl, aryloxy, carboxy, haloalkyl, hydroxy, cyano, nitroso, nitro, azido, trifluoromethyl, trifluoromethoxy, thio, alkylthio, arylthiol, alkylsulfonyl, alkylsulfinyl, dialkylaminosulfonyl, sulfonic acid, carboxylic acid, dialkylamino and dialkylamido. In some embodiments, representative substituted alkyl groups can be substituted from a set of groups including amino, hydroxy, cyano, carboxy, nitro, thio and alkoxy, but not including halogen groups.

The terms “halo,” “halogen,” or “halide” group, as used herein, by themselves or as part of another substituent, mean, unless otherwise stated, a fluorine, chlorine, bromine, or iodine atom.

The term “acyl” as used herein refers to a group containing a carbonyl moiety wherein the group is bonded via the carbonyl carbon atom. The carbonyl carbon atom is also bonded to another carbon atom, which can be part of a substituted or unsubstituted alkyl, alkenyl, alkynyl, aryl, cycloalkyl, heterocyclyl, group or the like.

The term “alkenyl” as used herein refers to substituted or unsubstituted straight chain, branched and cyclic, saturated mono-or bi-valent groups having at least one carbon-carbon double bond and from 2 to 20 carbon atoms, 10 to 20 carbon atoms, 12 to 18 carbon atoms, 6 to about 10 carbon atoms, 2 to 10 carbons atoms, 2 to 8 carbon atoms, 3 to 8 carbon atoms, 4 to 8 carbon atoms, 5 to 8 carbon atoms, 2 to 6 carbon atoms, 3 to 6 carbon atoms, 4 to 6 carbon atoms, 2 to 4 carbon atoms, or 2 to 3 carbon atoms. The double bonds can be trans or cis orientation. The double bonds can be terminal or internal. The alkenyl group can be attached via the portion of the alkenyl group containing the double bond, e.g., vinyl, propen-1-yl and buten-1-yl, or the alkenyl group can be attached via a portion of the alkenyl group that does not contain the double bond, e.g., penten-4-yl. Examples of mono-valent (C₂-C₂₀)-alkenyl groups include those with from 1 to 8 carbon atoms such as vinyl, propenyl, propen-1-yl, propen-2-yl, butenyl, buten-1-yl, buten-2-yl, sec-buten-1-yl, sec-buten-3-yl, pentenyl, hexenyl, heptenyl and octenyl groups. Examples of branched mono-valent (C₂-C₂₀)-alkenyl groups include isopropenyl, iso-butenyl, sec-butenyl, t-butenyl, neopentenyl, and isopentenyl. Examples of straight chain bi-valent (C₂-C₂₀)alkenyl groups include those with from 2 to 6 carbon atoms such as —CHCH—, —CHCHCH₂—, —CHCHCH₂CH₂—, and —CHCHCH₂CH₂CH₂—. Examples of branched bi-valent alkyl groups include —C(CH₃)CH— and —CHC(CH₃)CH₂—. Examples of cyclic alkenyl groups include cyclopentenyl, cyclohexenyl and cyclooctenyl. It is envisaged that alkenyl can also include masked alkenyl groups, precursors of alkenyl groups or other related groups. As such, where alkenyl groups are described it, compounds are also envisaged where a carbon-carbon double bond of an alkenyl is replaced by an epoxide or aziridine ring. Substituted alkenyl also includes alkenyl groups which are substantially tautomeric with a non-alkenyl group. For example, substituted alkenyl can be 2-aminoalkenyl, 2-alkylaminoalkenyl, 2-hydroxyalkenyl, 2-hydroxyvinyl, 2-hydroxypropenyl, but substituted alkenyl is also understood to include the group of substituted alkenyl groups other than alkenyl which are tautomeric with non-alkenyl containing groups. In some embodiments, alkenyl can be understood to include a combination of substituted and unsubstituted alkenyl. For example, alkenyl can be vinyl and substituted vinyl. For example, alkenyl can be vinyl and substituted (C₃-C₈)alkenyl. Alkenyl can also include substituted vinyl and unsubstituted (C₃-C₈)alkenyl. Representative substituted alkenyl groups can be substituted one or more times with any of the groups listed herein, for example, monoalkylamino, dialkylamino, cyano, acetyl, amido, carboxy, nitro, alkylthio, alkoxy, and halogen groups. As further example, representative substituted alkenyl groups can be substituted one or more fluoro, chloro, bromo, iodo, amino, amido, alkyl, alkoxy, alkylamido, alkenyl, alkynyl, alkoxycarbonyl, acyl, formyl, arylcarbonyl, aryloxycarbonyl, aryloxy, carboxy, haloalkyl, hydroxy, cyano, nitroso, nitro, azido, trifluoromethyl, trifluoromethoxy, thio, alkylthio, arylthiol, alkylsulfonyl, alkylsulfinyl, dialkylaminosulfonyl, sulfonic acid, carboxylic acid, dialkylamino and dialkylamido. In some embodiments, representative substituted alkenyl groups can be substituted from a set of groups including monoalkylamino, dialkylamino, cyano, acetyl, amido, carboxy, nitro, alkylthio and alkoxy, but not including halogen groups. Thus, in some embodiments, alkenyl can be substituted with a non-halogen group. In some embodiments, representative substituted alkenyl groups can be substituted with a fluoro group, substituted with a bromo group, substituted with a halogen other than bromo, or substituted with a halogen other than fluoro. For example, alkenyl can be 1-fluorovinyl, 2-fluorovinyl, 1,2-difluorovinyl, 1,2,2-trifluorovinyl, 2,2-difluorovinyl, trifluoropropen-2-yl, 3,3,3-trifluoropropenyl, 1-fluoropropenyl, 1-chlorovinyl, 2-chlorovinyl, 1,2-dichlorovinyl, 1,2,2-trichlorovinyl or 2,2-dichlorovinyl. In some embodiments, representative substituted alkenyl groups can be substituted with one, two, three or more fluoro groups or they can be substituted with one, two, three or more non-fluoro groups.

The term “alkynyl” as used herein, refers to substituted or unsubstituted straight and branched chain alkyl groups, except that at least one triple bond exists between two carbon atoms. Thus, alkynyl groups have from 2 to 50 carbon atoms, 2 to 20 carbon atoms, 10 to 20 carbon atoms, 12 to 18 carbon atoms, 6 to about 10 carbon atoms, 2 to 10 carbons atoms, 2 to 8 carbon atoms, 3 to 8 carbon atoms, 4 to 8 carbon atoms, 5 to 8 carbon atoms, 2 to 6 carbon atoms, 3 to 6 carbon atoms, 4 to 6 carbon atoms, 2 to 4 carbon atoms, or 2 to 3 carbon atoms. Examples include, but are not limited to ethynyl, propynyl, propyn-1-yl, propyn-2-yl, butynyl, butyn-1-yl, butyn-2-yl, butyn-3-yl, butyn-4-yl, pentynyl, pentyn-1-yl, hexynyl, Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “aryl” as used herein refers to substituted or unsubstituted univalent groups that are derived by removing a hydrogen atom from an arene, which is a cyclic aromatic hydrocarbon, having from 6 to 20 carbon atoms, 10 to 20 carbon atoms, 12 to 20 carbon atoms, 6 to about 10 carbon atoms or 6 to 8 carbon atoms. Examples of (C₆-C₂₀)aryl groups include phenyl, napthalenyl, azulenyl, biphenylyl, indacenyl, fluorenyl, phenanthrenyl, triphenylenyl, pyrenyl, naphthacenyl, chrysenyl, anthracenyl groups. Examples include substituted phenyl, substituted napthalenyl, substituted azulenyl, substituted biphenylyl, substituted indacenyl, substituted fluorenyl, substituted phenanthrenyl, substituted triphenylenyl, substituted pyrenyl, substituted naphthacenyl, substituted chrysenyl, and substituted anthracenyl groups. Examples also include unsubstituted phenyl, unsubstituted napthalenyl, unsubstituted azulenyl, unsubstituted biphenylyl, unsubstituted indacenyl, unsubstituted fluorenyl, unsubstituted phenanthrenyl, unsubstituted triphenylenyl, unsubstituted pyrenyl, unsubstituted naphthacenyl, unsubstituted chrysenyl, and unsubstituted anthracenyl groups. Aryl includes phenyl groups and also non-phenyl aryl groups. From these examples, it is clear that the term (C₆-C₂₀)aryl encompasses mono-and polycyclic (C₆-C₂₀)aryl groups, including fused and non-fused polycyclic (C₆-C₂₀)aryl groups.

The term “heterocyclyl” as used herein refers to substituted aromatic, unsubstituted aromatic, substituted non-aromatic, and unsubstituted non-aromatic rings containing 3 or more atoms in the ring, of which, one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. In some embodiments, heterocyclyl groups include heterocyclyl groups that include 3 to 8 carbon atoms (C₃-C₈), 3 to 6 carbon atoms (C₃-C₆) or 6 to 8 carbon atoms (C₆-C₈). A heterocyclyl group designated as a C₂-heterocyclyl can be a 5-membered ring with two carbon atoms and three heteroatoms, a 6-membered ring with two carbon atoms and four heteroatoms and so forth. Likewise, a C₄-heterocyclyl can be a 5-membered ring with one heteroatom, a 6-membered ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. Representative heterocyclyl groups include, but are not limited to piperidynyl, piperazinyl, morpholinyl, furanyl, pyrrolidinyl, pyridinyl, pyrazinyl, pyrimidinyl, triazinyl, thiophenyl, tetrahydrofuranyl, pyrrolyl, oxazolyl, imidazolyl, triazyolyl, tetrazolyl, benzoxazolinyl, and benzimidazolinyl groups. For example, heterocyclyl groups include, without limitation:

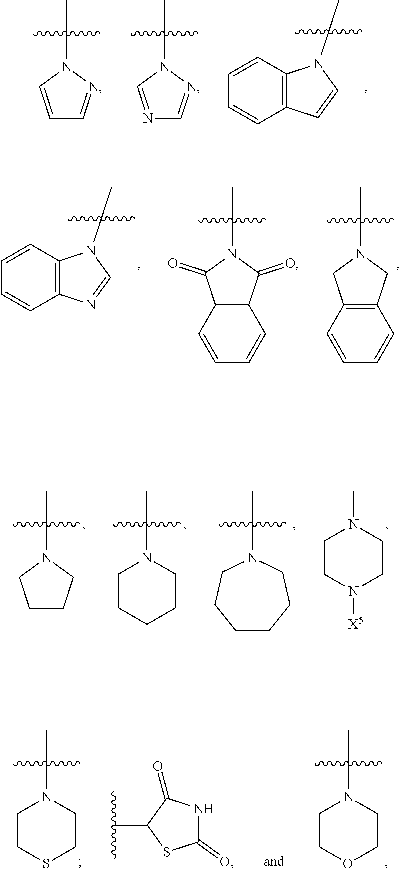

wherein X⁵ represents H, (C₁-C₂₀)alkyl, (C₆-C₂₀)aryl or an amine protecting group (e.g., a t-butyloxycarbonyl group) and wherein the heterocyclyl group can be substituted or unsubstituted. A nitrogen-containing heterocyclyl group is a heterocyclyl group containing a nitrogen atom as an atom in the ring. In some embodiments, the heterocyclyl is other than thiophene or substituted thiophene. In some embodiments, the heterocyclyl is other than furan or substituted furan.

The term “aralkyl” and “arylalkyl” as used herein refers to alkyl groups as defined herein in which a hydrogen or carbon bond of an alkyl group is replaced with a bond to an aryl group as defined herein. Representative aralkyl groups include benzyl, biphenylmethyl and phenylethyl groups and fused (cycloalkylaryl) alkyl groups such as 4-ethyl-indanyl. Aralkenyl groups are alkenyl groups as defined herein in which a hydrogen or carbon bond of an alkyl group is replaced with a bond to an aryl group as defined herein.

The term “substituted” as used herein refers to a group that is substituted with one or more groups including, but not limited to, the following groups: halogen (e.g., F, Cl, Br, and I), R, OR, ROH (e.g., CH2OH), OC(O)N(R)2, CN, NO, NO2, ONO2, azido, CF3, OCF3, methylenedioxy, ethylenedioxy, (C3-C20)heteroaryl, N(R)2, Si(R)3, SR, SOR, SO2R, SO2N(R)2, SO3R, P(O)(OR)2, OP(O)(OR)2, C(O)R, C(O)C(O)R, C(O)CH2C(O)R, C(S)R, C(O)OR, OC(O)R, C(O)N(R)2, C(O)N(R)OH, OC(O)N(R)2, C(S)N(R)2, (CH2)0-2N(R)C(O)R, (CH2)0-2N(R)N(R)2, N(R)N(R)C(O)R, N(R)N(R)C(O)OR, N(R)N(R)CON(R)2, N(R)SO2R, N(R)SO2N(R)2, N(R)C(O)OR, N(R)C(O)R, N(R)C(S)R, N(R)C(O)N(R)2, N(R)C(S)N(R)2, N(COR)COR, N(OR)R, C(═NH)N(R)2, C(O)N(OR)R, or C(═NOR)R wherein R can be hydrogen, (C1-C20)alkyl, (C6-C20)aryl, heterocyclyl or polyalkylene oxide groups, such as polyalkylene oxide groups of the formula —(CH2CH20)f-R—OR, —(CH2CH2CH2O)g-R—OR, —(CH2CH2O)f(CH2CH2CH2O)g-R—OR each of which can, in turn, be substituted or unsubstituted and wherein f and g are each independently an integer from 1 to 50 (e.g., 1 to 10, 1 to 5, 1 to 3 or 2 to 5). Substituted also includes a group that is substituted with one or more groups including, but not limited to, the following groups: fluoro, chloro, bromo, iodo, amino, amido, alkyl, hydroxy, alkoxy, alkylamido, alkenyl, alkynyl, alkoxycarbonyl, acyl, formyl, arylcarbonyl, aryloxycarbonyl, aryloxy, carboxy, haloalkyl, hydroxy, cyano, nitroso, nitro, azido, trifluoromethyl, trifluoromethoxy, thio, alkylthio, arylthiol, alkylsulfonyl, alkylsulfinyl, dialkylaminosulfonyl, sulfonic acid, carboxylic acid, dialkylamino and dialkylamido. Where there are two or more adjacent substituents, the substituents can be linked to form a carbocyclic or heterocyclic ring. Such adjacent groups can have a vicinal or germinal relationship, or they can be adjacent on a ring in, e.g., an ortho-arrangement. Each instance of substituted is understood to be independent. For example, a substituted aryl can be substituted with bromo and a substituted heterocycle on the same compound can be substituted with alkyl. It is envisaged that a substituted group can be substituted with one or more non-fluoro groups. As another example, a substituted group can be substituted with one or more non-cyano groups. As another example, a substituted group can be substituted with one or more groups other than haloalkyl. As yet another example, a substituted group can be substituted with one or more groups other than tert-butyl. As yet a further example, a substituted group can be substituted with one or more groups other than trifluoromethyl. As yet even further examples, a substituted group can be substituted with one or more groups other than nitro, other than methyl, other than methoxymethyl, other than dialkylaminosulfonyl, other than bromo, other than chloro, other than amido, other than halo, other than benzodioxepinyl, other than polycyclic heterocyclyl, other than polycyclic substituted aryl, other than methoxycarbonyl, other than alkoxycarbonyl, other than thiophenyl, or other than nitrophenyl, or groups meeting a combination of such descriptions. Further, substituted is also understood to include fluoro, cyano, haloalkyl, tert-butyl, trifluoromethyl, nitro, methyl, methoxymethyl, dialkylaminosulfonyl, bromo, chloro, amido, halo, substituted aryl, benzodioxepinyl, polycyclic heterocyclyl, polycyclic methoxycarbonyl, alkoxycarbonyl, thiophenyl, and nitrophenyl groups.

### Hosts

Terpenes, including diterpenes and terpenoids, can be made in a variety of host organisms in vivo. In some cases, the enzymes described herein can be made in host cells, and those enzymes can be extracted from the host cells for use in vitro. As used herein, a “host” means a cell, tissue or organism capable of replication. The host can have an expression cassette or expression vector that can include a nucleic acid segment encoding an enzyme that is involved in the biosynthesis of terpenes.

The term “host cell”, as used herein, refers to any prokaryotic or eukaryotic cell that can be transformed with an expression cassettes or vector carrying the nucleic acid segment encoding an enzyme that is involved in the biosynthesis of one or more terpenes or terpenoids. The host cells can, for example, be a plant, bacterial, insect, or yeast cell. Expression cassettes encoding biosynthetic enzymes can be incorporated or transferred into a host cell to facilitate manufacture of the enzymes described herein or the terpene, diterpene, or terpenoid products of those enzymes. The host cells can be present in an organism. For example, the host cells can be present in a host such as a microorganism, fungus, or plant. As illustrated herein, the host can be a plant cell such as a Nicotiana benthamiana host cell.

### Expression of Enzymes

Also described herein are expression systems that include at least one expression cassette (e.g., expression vectors or transgenes) that encode one or more of the enzyme(s) described herein. For example, the expression systems can also include one or more expression cassettes any of the monoterpene synthase, diterpene synthase, sesquiterpene synthase, sesterterpene synthase, triterpene synthase, tetraterpene synthase, polyterpene synthase, transcription factor, cis-prenyl transferase, terpene synthase, cytochrome P450 (CYP71D616), cytochrome P450 reductase, 1-deoxy-D-xylulose 5-phosphate synthase (DXS), 1-deoxy-D-xylulose 5-phosphate-reducto-isomerase, cytidine 5′-diphosphate-methylerythritol (CDP-ME) synthetase (IspD), 2-C-methyl-d-erythritol 2,4-cyclodiphosphate synthase (IspF), HMG-COA synthase, HMG-COA reductase (HMGR), mevalonic acid kinase (MVK), phosphomevalonate kinase (PMK), mevalonate-5-diphosphate decarboxylase (MPD), isopentenyl diphosphate isomerase, abietadiene synthase (ABS), farnesylpyrophosphate synthase (FPPS), or squalene synthase (SQS), LDSP-protein fusions, or enzymes that facilitate production of terpenoids, terpene precursors, terpene building blocks, or products derived from terpenoids.

Nucleic acids encoding the enzymes can have sequence modifications. For example, nucleic acid sequences described herein can be modified to more optimally express the enzymes. Hence, the nucleic acid segment encoding the enzymes can be optimized to improve expression in different host cells. Most amino acids can be encoded by more than one codon, but when an amino acid is encoded by more than one codon, the codons are referred to as degenerate codons. A listing of degenerate codons is provided in Table 1B below.

TABLE 1B
Degenerate Amino Acid Codons

| Amino Acid | Three Nucleotide Codon |
|---|---|
| Ala/A | GCT, GCC, GCA, GCG |
| Arg/R | CGT, CGC, CGA, CGG, AGA, AGG |
| Asn/N | AAT, AAC |
| Asp/D | GAT, GAC |
| Cys/C | TGT, TGC |
| Gln/Q | CAA, CAG |
| Glu/E | GAA, GAG |
| Gly/G | GGT, GGC, GGA, GGG |
| His/H | CAT, CAC |
| Ile/I | ATT, ATC, ATA |
| Leu/L | TTA, TTG, CTT, CTC, CTA, CTG |
| Lys/K | AAA, AAG |
| Met/M | ATG |
| Phe/F | TTT, TTC |
| Pro/P | CCT, CCC, CCA, CCG |
| Ser/S | TCT, TCC, TCA, TCG, AGT, AGC |
| Thr/T | ACT, ACC, ACA, ACG |
| Trp/W | TGG |
| Tyr/Y | TAT, TAG |
| Val/V | GTT, GTC, GTA, GTG |
| START | ATG |
| STOP | TAG, TGA, TAA |

Different organisms may translate different codons more or less efficiently (e.g., because they have different ratios of tRNAs) than other organisms. Hence, when some amino acids can be encoded by several codons, a nucleic acid segment can be designed to optimize the efficiency of expression of an enzyme by using codons that are preferred by an organism of interest. For example, the nucleotide coding regions of the enzymes described herein can be codon optimized for expression in various microorganisms, fungi, or plant species.

An optimized nucleic acid can have less than 100%, less than 99%, less than 98%, less than 97%, less than 95%, or less than 94%, or less than 93%, or less than 92%, or less than 91%, or less than 90%, or less than 89%, or less than 88%, or less than 85%, or less than 83%, or less than 80%, or less than 75% nucleic acid sequence identity to a corresponding non-optimized (e.g., a non-optimized parental or wild type enzyme nucleic acid) sequence. Nucleic acid segment(s) encoding one or more enzyme(s) can therefore have one or more nucleotide deletions, insertions, replacements, or substitutions.

The nucleic acid segments encoding one or more enzyme can be operably linked to a promoter, which provides for expression of mRNA from the nucleic acid segments. The promoter is typically a promoter functional in a microorganism, fungus or plant. A nucleic acid segment encoding one or more enzyme is operably linked to the promoter, for example, when it is located downstream from the promoter. The combination of a coding region for an enzyme operably linked to a promoter forms an expression cassette, which can include other elements and regulatory sequences as well.

Promoter regions are typically found in the flanking DNA upstream from the coding sequence in both the prokaryotic and eukaryotic cells. A promoter sequence provides for regulation of transcription of the downstream gene sequence and typically includes from about 50 to about 2,000 nucleotide base pairs. Promoter sequences can also contain regulatory sequences such as enhancer sequences that can influence the level of gene expression. Some isolated promoter sequences can provide for gene expression of heterologous DNAs, that is a DNA different from the native or homologous DNA.

Promoter sequences are also known to be strong or weak, or inducible. A strong promoter provides for a high level of gene expression, whereas a weak promoter provides for a very low level of gene expression. An inducible promoter is a promoter that provides for the turning on and off of gene expression in response to an exogenously added agent, or to an environmental or developmental stimulus. For example, a bacterial promoter such as the Ptac promoter can be induced to varying levels of gene expression depending on the level of isopropyl-beta-D-thiogalactoside added to the transformed cells. Promoters can also provide for tissue specific or developmental regulation. An isolated promoter sequence that is a strong promoter for heterologous DNAs is often advantageous because it provides for a sufficient level of gene expression for easy detection and selection of transformed cells and provides for a high level of gene expression when desired.

Examples of prokaryotic promoters that can be used include, but are not limited to, SP6, T7, T5, tac, bla, trp, gal, lac, or maltose promoters. Examples of eukaryotic promoters that can be used include, but are not limited to, constitutive promoters, e.g., viral promoters such as CMV, SV40 and RSV promoters, as well as regulatable promoters, e.g., an inducible or repressible promoter such as the tet promoter, the hsp70 promoter and a synthetic promoter regulated by CRE.

Examples of plant promoters include the CaMV 35S promoter (Odell et al., Nature. 313:810-812 (1985)), or others such as CaMV 19S (Lawton et al., Plant Molecular Biology. 9:315-324 (1987)), nos (Ebert et al., Proc. Natl. Acad. Sci. USA. 84:5745-5749 (1987)), Adh1 (Walker et al., Proc. Natl. Acad. Sci. USA. 84:6624-6628 (1987)), sucrose synthase (Yang et al., Proc. Natl. Acad. Sci. USA. 87:4144-4148 (1990)), α-tubulin, ubiquitin, actin (Wang et al., Mol. Cell. Biol. 12:3399 (1992)), cab (Sullivan et al., Mol. Gen. Genet. 215:431 (1989)), PEPCase (Hudspeth et al., Plant Molecular Biology. 12:579-589 (1989)) or those associated with the R gene complex (Chandler et al., The Plant Cell. 1:1175-1183 (1989)). Further suitable promoters include a CYP71D16 trichome-specific promoter and the CBTS (cembratrienol synthase) promotor, cauliflower mosaic virus promoter, the Z10 promoter from a gene encoding a 10 kD zein protein, a Z27 promoter from a gene encoding a 27 kD zein protein, the plastid rRNA-operon (rrn) promoter, inducible promoters, such as the light inducible promoter derived from the pea rbcS gene (Coruzzi et al., EMBO J. 3:1671 (1971)), RUBISCO-SSU light inducible promoter (SSU) from tobacco and the actin promoter from rice (McElroy et al., The Plant Cell. 2:163-171 (1990)). Other promoters that are useful can also be employed.

Examples of leaf-specific promoters include the promoter from the Populus ribulose-1,5-bisphosphate carboxylase small subunit gene (Wang et al. Plant Molec Biol Reporter 31 (1): 120-127 (2013)), the promoter from the Brachypodium distachyon sedoheptulose-1,7-bisphosphatase (SBPase-p) gene (Alotaibi et al. Plants 7 (2): 27 (2018)), the fructose-1,6-bisphosphate aldolase (FBPA-p) gene from Brachypodium distachyon (Alotaibi et al. Plants 7 (2): 27 (2018)), and the photosystem-Il promoter (CAB2-p) of the rice (Oryza sativa L.) light-harvest chlorophyll a/b binding protein (CAB) (Song et al. J Am Soc Hort Sci 132(4): 551-556 (2007)). Additional promoters that can be used include those available in expression databases, see for example, website bar.utoronto.ca/eplant/which includes poplar or heterologous promoters from Arabidopsis (for example from AT2G26020/PDF1.2b or AT5G44420/LCR77).

Alternatively, novel tissue specific promoter sequences may be employed. cDNA clones from a particular tissue can be isolated and those clones which are expressed specifically in that tissue can be identified, for example, using Northern blotting. Preferably, the gene isolated is not present in a high copy number but is relatively abundant in specific tissues. The promoter and control elements of corresponding genomic clones can then be localized using techniques well known to those of skill in the art.

Plant plastid originated promoters can also be used, for example, to improve expression in plastids, for example, a rice clp promoter, or tobacco rrn promoter. Chloroplast-specific promoters can also be utilized for targeting the foreign protein expression into chloroplasts. For example, the 16S ribosomal RNA promoter (Prrn) like psbA and atpA gene promoters can be used for chloroplast transformation.

A nucleic acid encoding one or more enzyme can be combined with the promoter by standard methods to yield an expression cassette, for example, as described in Sambrook et al. (Molecular Cloning: A Laboratory Manual. Second Edition (Cold Spring Harbor, NY: Cold Spring Harbor Press (1989); Molecular Cloning: A Laboratory Manual. Third Edition (Cold Spring Harbor, NY: Cold Spring Harbor Press (2000)). Briefly, a plasmid containing a promoter such as the 35S CaMV promoter or the CYP71D16 trichome-specific promoter can be constructed as described in Jefferson (Plant Molecular Biology Reporter 5:387-405 (1987)) or obtained from Clontech Lab in Palo Alto, California (e.g., pBI121 or pBI221). Typically, these plasmids are constructed to have multiple cloning sites having specificity for different restriction enzymes downstream from the promoter.

The expression cassette or vector can include nucleic acid sequence encoding a marker product. This marker product is used to determine if the gene has been delivered to the cell and once delivered is being expressed. Marker genes can include the E. coli lacZ gene which encodes β-galactosidase, and green fluorescent protein. In some embodiments the marker can be a selectable marker. When such selectable markers are successfully transferred into a host cell, the transformed host cell can survive if placed under selective pressure. There are two widely used distinct categories of selective regimes. The first category is based on a cell's metabolism and the use of a mutant cell line which lacks the ability to grow independent of a supplemented media. The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)).

The expression cassettes can be within vectors such as plasmids, viral vectors, viral nucleic acids, phage nucleic acids, phages, cosmids, or artificial chromosomes.

Transfer of the expression cassettes or vectors into host cells can be by methods available in the art and readily adaptable for use in the method described herein. Expression cassettes and vectors can be incorporated into host cells, for example, calcium-mediated transformation, electroporation, microinjection, lipofection, particle bombardment, chemical transfectants, physico-mechanical methods such as electroporation, or direct diffusion of DNA.

### Methods

Methods are described herein that are useful for synthesizing terpenoids and products made from terpenoids. The methods can involve contacting one or more of the substrates described herein with one or more enzymes capable of synthesizing at least one terpene to produce a terpenoid product. In some cases, the methods can involve incubating one or more of the substrates described herein with a population of host cells having a at least one heterologous expression cassette or expression vector that can express one or more enzymes capable of synthesizing at least one terpenoid product. The enzymes capable of synthesizing at least one terpenoid product can be referred to as a primary enzyme. The methods can also involve contacting the terpenoid product with a secondary enzyme that can modify the terpenoid product into another useful product.

For example, one method can involve contacting one or more of the substrates described herein with one or more enzymes capable of synthesizing at least one terpene to produce a terpenoid product.

For example, another method can involve (a) incubating a population of host cells or host tissue that includes one or more expression cassettes (or vectors) that have a promoter operably linked to a nucleic acid segment encoding an enzyme capable of synthesizing at least one terpene; and (b) isolating at least one terpenoid product from the population of host cells or the host tissue.

The enzymes can be any of the enzymes described herein. For example, the enzymes can be a monoterpene synthase, diterpene synthase, sesquiterpene synthase, sesterterpene synthase, triterpene synthase, tetraterpene synthase, or polyterpene synthase. Enzymes used for modifying a terpenoid product (e.g., secondary enzymes) can include one or more transcription factor, cis-prenyl transferase, terpene synthase, cytochrome P450 (CYP71D616), cytochrome P450 reductase, 1-deoxy-D-xylulose 5-phosphate synthase (DXS), 1-deoxy-D-xylulose 5-phosphate-reducto-isomerase, cytidine 5′-diphosphate-methylerythritol (CDP-ME) synthetase (IspD), 2-C-methyl-d-erythritol 2,4-cyclodiphosphate synthase (IspF), geranylgeranyl diphosphate synthase (GGDPS), HMG-COA synthase, HMG-CoA reductase (HMGR), mevalonic acid kinase (MVK), phosphomevalonate kinase (PMK), mevalonate-5-diphosphate decarboxylase (MPD), isopentenyl diphosphate isomerase (IDI), abietadiene synthase (ABS), farnesylpyrophosphate synthase (FPPS), ribulose bisphosphate carboxylase, squalene synthase (SQS), patchoulol synthase, or WRI1 protein; and (b) isolating useful products from the population of host cells, the host plant's cells, or the host tissue. In some cases, a combination of enzymes, transcription factors, and lipid droplet proteins can be expressed in host cells, host plant, or host tissues.

### Definitions

As used herein, the singular forms “a,” “an,” and “the” are intended to include the plural forms as well, unless the context clearly indicates otherwise. Also, as used herein, “and/or” refers to, and encompasses, any and all possible combinations of one or more of the associated listed items. Unless otherwise defined, all terms, including technical and scientific terms used in the description, have the same meaning as commonly understood by one of ordinary skill in the art to which this invention pertains.

The term “about”, as used herein, can allow for a degree of variability in a value or range, for example, within 10%, within 5%, or within 1% of a stated value or of a stated limit of a range.

The term “enzyme” or “enzymes”, as used herein, refers to a protein catalyst capable of catalyzing a reaction. Herein, the term does not mean only an isolated enzyme, but also includes a host cell expressing that enzyme. Accordingly, the conversion of A to B by enzyme C should also be construed to encompass the conversion of A to B by a host cell expressing enzyme C.

The term “heterologous” when used in reference to a nucleic acid refers to a nucleic acid that has been manipulated in some way. For example, a heterologous nucleic acid includes a nucleic acid from one species introduced into another species. A heterologous nucleic acid also includes a nucleic acid native to an organism that has been altered in some way (e.g., mutated, added in multiple copies, linked to a non-native promoter or enhancer sequence, etc.). Heterologous nucleic acids can include cDNA forms of a nucleic acid; the cDNA may be expressed in either a sense (to produce mRNA) or anti-sense orientation (to produce an anti-sense RNA transcript that is complementary to the mRNA transcript). For example, heterologous nucleic acids can be distinguished from endogenous plant nucleic acids in that the heterologous nucleic acids are typically joined to nucleic acids comprising regulatory elements such as promoters that are not found naturally associated with the natural gene for the protein encoded by the heterologous gene. Heterologous nucleic acids can also be distinguished from endogenous plant nucleic acids in that the heterologous nucleic acids are in an unnatural chromosomal location or are associated with portions of the chromosome not found in nature (e.g., the heterologous nucleic acids are expressed in tissues where the gene is not normally expressed).

The terms “identical” or percent “identity”, as used herein, in the context of two or more nucleic acids or polypeptide sequences, refer to two or more sequences or subsequences that are the same or have a specified percentage of amino acid residues or nucleotides that are the same (e.g., 75% identity, 80% identity, 85% identity, 90% identity, 95% identity, 97% identity, 98% identity, 99% identity, or 100% identity in pairwise comparison). Sequence identity can be determined by comparison and/or alignment of sequences for maximum correspondence over a comparison window, or over a designated region as measured using a sequence comparison algorithm, or by manual alignment and visual inspection. The percentage is calculated by determining the number of positions at which the identical nucleic acid base or amino acid residue occurs in both sequences to yield the number of matched positions, dividing the number of matched positions by the total number of positions in the window of comparison and multiplying the results by 100 to yield the percentage of sequence identity. A “reference sequence” is a defined sequence used as a basis for a sequence comparison; a reference sequence may be a subset of a larger sequence.

As used herein, a “native” nucleic acid or polypeptide means a DNA, RNA, or amino acid sequence or segment thereof that has not been manipulated in vitro, i.e., has not been isolated, purified, amplified and/or modified.

The terms “in operable combination,” “in operable order,” and “operably linked” refer to the linkage of nucleic acid sequences in such a manner that a nucleic acid molecule capable of directing the transcription of a coding region (e.g., gene) and/or the synthesis of a desired protein molecule is produced. The term also refers to the linkage of amino acid sequences in such a manner so that a functional protein is produced.

As used herein the term “terpene” includes any type of terpene or terpenoid, including for example any monoterpene, diterpene, sesquiterpene, sesterterpene, triterpene, tetraterpene, polyterpene, and any mixture thereof.

As used herein, the term “wild-type” when made in reference to a gene refers to a functional gene common throughout an outbred population. As used herein, the term “wild-type” when made in reference to a gene product refers to a functional gene product common throughout an outbred population. A functional wild-type gene is that which is most frequently observed in a population and is thus arbitrarily designated the “normal” or “wild-type” form of the gene.

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

### EXAMPLES

The present disclosure can be better understood by reference to the following examples which are offered by way of illustration and which are described in Plant J. 2020 November; 104(3):693-705, which is incorporated by reference as if fully set forth herein. The disclosure is not limited to the examples given herein.

### Materials and Methods

### Plant Material, RNA Isolation and cDNA Synthesis, and Metabolite Analysis

Leucophyllum frutescens plants were obtained from Stokes Tropicals (Homestead, FL, USA) and grown in a greenhouse under ambient photoperiod and 24° C. day/17° C. night temperatures. Total RNA from flower, leaf, and root tissues was extracted following methods described in Plant Physiol. 157, 1677-1695 (2011) using the Spectrum™ Plant Total RNA Kit (Sigma-Aldrich, St. Louis, MO, USA). RNA extraction was followed by DNase I digestion using DNA-free™ DNA Removal Kit (ThermoFisher Scientific). Total RNA was assessed for quantity and integrity by Qubit™ (ThermoFisher Scientific) and RNA-nano assays (Agilent Bioanalyzer 2100), prior to whole transcriptome sequencing (Novogene, Sacramento, CA, USA) First-strand cDNA was synthesized from 2 μg of root total RNA using SuperScript III (Invitrogen). For GC-MS-based metabolomics, approximately 1 g of root, leaf, or flower tissue was extracted in 1 mL MTBE for 3 hours and analyzed by GC-MS with the same method described below for analysis of enzyme assays.

### L. frutescens and E. serrulata De Novo Transcriptome Assembly and Analysis

RNA-seq data were obtained through tissue-specific RNA sequencing on an Illumina HiSeq 4000 for L. frutescens and the NCBI Sequence Read Archive (https://www.ncbi.nlm.nih.gov/sra (ERX1321488)) for E. serrulata Phytochemistry 136, 15-22 (2017). Quality of sequencing data was checked with FastQC (v0.11.4), and adapters were trimmed with Trimmomatic (v0.39; Bioinformatics 30, 2114-2120 (2014). A transcriptome was assembled with Trinity (v2.8.4; Nat Biotechnol 29, 644-652 (2011)), expression levels calculated with Salmon (v.0.11.2; Nat. Methods 14, 417-419 (2017)), and open reading frames picked out with TransDecoder (v5.5.0; Nat Protoc 8, 1494-1512 (2013)). A BLAST (v2.7.1+) search against reference databases of respective enzyme families (Dataset S1) was done to pick out candidates. Phylogenetic trees were made with Clustal Omega (v1.2.4; Mol. Syst. Biol. 7, 539 (2011)) and RAXML (v8.0.0; Bioinformatics 30, 1312-1313 (2014)) and visualized with Interactive Tree of Live (Nucleic Acids Res 47, W256-W259 (2019)). Plastidial transit peptides were predicted between TargetP (v 1.1; Journal of Molecular Biology 300, 1005-1016 (2000)) and sequence alignments with Clustal Omega (v1.2.4; Mol. Syst. Biol. 7, 539 (2011)).

### Cloning and Sources of Genes Used

Synthetic oligonucleotides, GenBank accession numbers, and sequences of each enzyme characterized in this study are listed in Dataset S1. Candidate enzymes were PCR-amplified from root cDNA, and coding sequences were cloned through In-Fusion cloning into the plant expression vector pEAQ-HT (Plant Biotechnol. J. 7, 682-693 (2009)) for transient expression assays in N. benthamiana, or into pET-28b(+) for expression in E. coli. LfTPS1 and LfTPS2 were cloned into pET-28b(+) as N-terminal truncated constructs omitting the first 23 amino acid residues, removing their putative transit peptides. For in vitro assays, constructs for PvTPS4, PvTPS5, and PvHVS(Δ43) in pET-28b(+) made in New Phytologist 223, 323-335 (2019b) were used as positive controls. For in vivo E. coli assays, the same truncated LfTPS constructs described above were used. TPS constructs were co-transformed with pIRS (Applied Microbiology and Biotechnology, 85(6), 1893-1906 (2010)) and pNN (New Phytologist 223, 323-335 (2019b)).

For all assays in N. benthamiana, full-length candidates were cloned into PEAQ-HT. For cytosolic tests, TPS candidates were co-expressed with Euphorbia lathyris HMGR and Methanothermobacter thermautotrophicus GGPPS (Sadre et al. 2019) in the pEarlygate vector (The Plant Journal, 45(4), 616-629 (2006)). As a positive control for cytosolic tests, an N-terminal truncated construct of PvHVS (PvHVS(Δ43)) was cloned into pEAQ-HT in this study. For plastidial tests, each candidate was coexpressed with Coleus forskohlii DXS (Angewandte Chemie International Edition 55, 2142-2146 (2016)) in pEarlygate. TPS candidate tests involved either co-expression of C. forskohlii GGPPS (Angewandte Chemie International Edition 55, 2142-2146 (2016)) in pEarlygate or Solanum lycopersicum CPT2 in PEAQ-HT (New Phytologist 223, 323-335 (2019b)), with a full-length construct of PvHVS in PEAQ-HT as a positive control (New Phytologist 223, 323-335 (2019b)).

### In Vitro Assays

TPS expression and purification was carried out as described in New Phytologist 223, 323-335 (2019b). LfTPS1 and LfTPS2 constructs in pET-28b(+) were transformed into the E. coli C41 OverExpress strain. Primary cultures (5 mL LB plus 50 μg/mL kanamycin) were grown overnight 37° C., and 1 mL was used to inoculate a bulk culture (100 mL TB plus 50 μg/mL kanamycin). This culture was grown to an OD₆₀₀ of 0.6 at 37° C., and expression was induced with 0.2 mM IPTG. Expression was carried out overnight at 17° C., cells were collected by centrifugation, and resuspended in Buffer A (20 mM HEPES, pH 7.2, 25 mM imidazole, 500 mM NaCl, 5% (v/v) glycerol) plus 10 μL/ml protease inhibitor cocktail (Sigma) and 0.1 mg/ml lysozyme (VWR). Cells were lysed by sonication and centrifuged at 11,000×g for 30 min. Supernatants were loaded onto Ni-NTA columns (His Spin-Trap; GE Healthcare) preequilibrated with Buffer A, washed with two column volumes of Buffer A, and protein was eluted with Buffer B (Buffer A with 350 mM imidazole). Samples were de-salted with a PD MidiTrap G-25 column (GE Healthcare) preequilibrated with Buffer C (20 mM HEPES, pH 7.2, 1 mM MgCl₂, 350 mM NaCl, and 5% (v/v) glycerol). Purified enzymes were frozen in liquid nitrogen and stored at −80° C. prior to in vitro assays.

In vitro assays were carried out with 1 μM enzyme and 30 μM substrate (GPP, FPP, or GGPP; Cayman Chemical) in 750 μL Buffer D (50 mM HEPES, pH 7.2, 7.5 mM MgCl₂, and 5% (v/v) glycerol), with 500 uL hexane overlay. Reactions were carried out for 16 hours at 30° C., vortexed to extract products, and centrifuged to re-separate the aqueous and organic layers. The organic layer was directly removed for GC-MS analysis.

### Transient Expression in N. benthamiana

Transient expression assays in N. benthamiana were carried out as described earlier (J. Biol. Chem., jbc.RA118.006025 (2019a)). N. benthamiana plants were grown for 5 weeks in a controlled growth room under 16 h light (24° C.) and 8 h dark (17° C.) cycle before infiltration. Constructs of candidates in PEAQ and others used for co-expression were separately transformed into Agrobacterium tumefaciens strain LBA4404. Cultures were grown overnight at 30° C. in 10 mL LB plus 50 μg/mL kanamycin and 50 μg/mL rifampicin, collected by centrifugation, and washed with 10 mL water twice. Cells were resuspended and diluted to an OD₆₀₀ of 1.0 in water plus 200 μM acetosyringone and incubated at 30° C. for 2-3 hours. Separate cultures were mixed in a 1:1 ratio for each combination of enzyme tested (e.g. for leubethanol production, equal volumes of cultures were mixed harboring CfDXS, LfCPT1, LfTPS1, and CYP71D616). Mixed cultures were infiltrated with a syringe into the abaxial side of N. benthamiana leaves, and plants were returned to the controlled growth room for 5 days. Approximately 200 mg fresh weight from infiltrated leaves was extracted with 1 mL hexane overnight at 18° C., plant material was collected by centrifugation, and the organic phase was removed for GC-MS analysis.

### E. coli In Vivo Assays

For in vivo E. coli assays, an engineered E. coli system (J. Am. Chem. Soc. 129, 6684-6685 (2007)) was used. LfTPS1 (Δ23) and LfTPS2 (Δ23) were co-transformed with pIRS and pNN and grown overnight at 37° C. in 5 mL LB plus 25 μg/mL kanamycin, 17 μg/mL chloramphenicol, and 25 μg/mL streptomycin. A culture of 10 mL TB including the same antibiotics (same concentrations) was inoculated with 100 μL of the overnight culture and grown to an OD600 of 0.6 at 37° C. The incubation temperature was lowered to 16° C. for 1 hour, expression was induced with 0.5 mM IPTG, and cultures were supplemented with 1 mM MgCl₂ and 40 mM pyruvate. Cultures were incubated at 16° C. for an additional 60 hours before extraction with an equal volume of hexane and 2% (v/v) EtOH. The organic phase was separated by centrifugation and analyzed by GC-MS.

### Dihydroserrulatene Production Scale-Up and NMR

To generate enough of the major LfTPS1 product (dihydroserrulatene) for NMR analysis, production in the E. coli system was carried out as detailed above, scaled up to 1 L. Following extraction, the organic layer was separated by centrifugation, concentrated under N₂ gas, and analyzed by GC-MS to confirm the presence of the LfTPS1 product. This product was purified by silica gel flash column chromatography with a mobile phase of 10% ethyl acetate in hexane. NMR spectra were measured on an Agilent DirectDrive2 500 MHz spectrometer using CDCl₃ as the solvent. CDCl₃ peaks were referenced to 7.26 and 77.00 ppm for ¹H and ¹³C spectra, respectively.

### GC-MS

All GC-MS analyses were performed on an Agilent 7890A GC with an Agilent VF-5 ms column (30 m×250 μm×0.25 μm, with 10m EZ-Guard) and an Agilent 5975C detector. The inlet was set to 250° C. splitless injection of 1 μL, He carrier gas (1 ml/min), and the detector was activated following a 3 min solvent delay. All assays and tissue analysis, with the exception of in vitro assays against GPP, used the following method: temperature ramp start 40° C., hold 1 min, 40° C./min to 200° C., hold 4.5 min, 20° C./min to 240° C., 10° C./min to 280° C.; 40° C./min to 320° C.; hold 5 min (3 min hold for in vitro assays). For in vitro assays against GPP, the following method was used: temperature ramp start 40° C.; 10° C./min to 180° C.; 40° C./min to 320° C.; hold 3 min.

### Homology Modeling

Homology models for LfCPT1 were generated using I-TASSER (v. 5.1; Nat. Methods 12, 7-8 (2015)) with either Solanum habrochaites (Z-Z)-FPPS (PDB ID: 5HXN; ACS Omega 2, 930-936 (2017)) or LiLPPS (PDB ID: 5HC6; Angewandte Chemie International Edition 55, 4721-4724 (2016)) as the template structure. Figures were generated in PyMOL (v2.3).

### Data Availability

RNA-seq data for L. frutescens has been submitted to the NCBI Sequence Read Archive (SRA) under the accession numbers SRX8371655 (root) and SRX8371656 (flower). GenBank accession numbers for nucleotide sequences of all enzymes tested in this study are as follows: LfTPS1: MT136608; LfTPS2: MT136609; LfCPT1: MT136610; LfCPT2: MT136611; LfCPT3: MT136612; CYP706G22: MT136613; CYP76A112: MT136614; CYP736A294: MT136615; CYP736A295: MT136616; CYP71D615: MT136617; CYP71D616: MT136618 EsTPS1: MT136619. Additional L. frutescens class I TPS candidates which were cloned but not characterized: LfTPS3: MT521506; LfTPS5: MT521507; LfTPS6: MT521505; LfTPS7: MT521508; LfTPS8a: MT521515; LfTPS8b: MT521516; LfTPS9: MT521509; LfTPS10: MT521511; LfTPS11: MT521510; LfTPS12a: MT521512; LfTPS12b: MT521513; LfTPS13: MT521514.

### Example 1: Accumulation of Leubethanol Guided Tissue-Specific RNA Sequencing

To begin the search for the biosynthetic pathway to leubethanol, the inventor(s) took advantage of its tissue-specific accumulation in L. frutescens. Previous work on the medicinal properties of this species has shown that root extracts were most potent against multi-drug-resistant tuberculosis, while leaves showed some activity and flowers showed none (Journal of Ethnopharmacology 109, 435-441 (2007)). To confirm the tissue-specific accumulation of leubethanol, extracts of the leaves, roots, and flowers were analyzed by GC-MS. Leubethanol was found to accumulate in both root and leaf tissue, while none was detected in flower tissue. Consequently, we isolated and sequenced RNA from both the roots and flowers to allow for comparative transcriptomics between tissue types. Serrulatane diterpenoids are also found in the closely related Eremophila genus. Phytochemistry 35, 7-33 (1993). RNA-seq data are publicly available from the leaves of E. serrulata (SRA: ERX1321488; (Phytochemistry 136, 15-22 (2017)) and serrulatanes are known to accumulate in this tissue (Ndi, 2007b). These data were also included to allow for comparison between genera.

### Example 2: Identification of TPS Candidates from L. frutescens

The search began by identifying TPS candidates from L. frutescensthrough a homologybased search of our transcriptomic data against a reference set of TPSs.

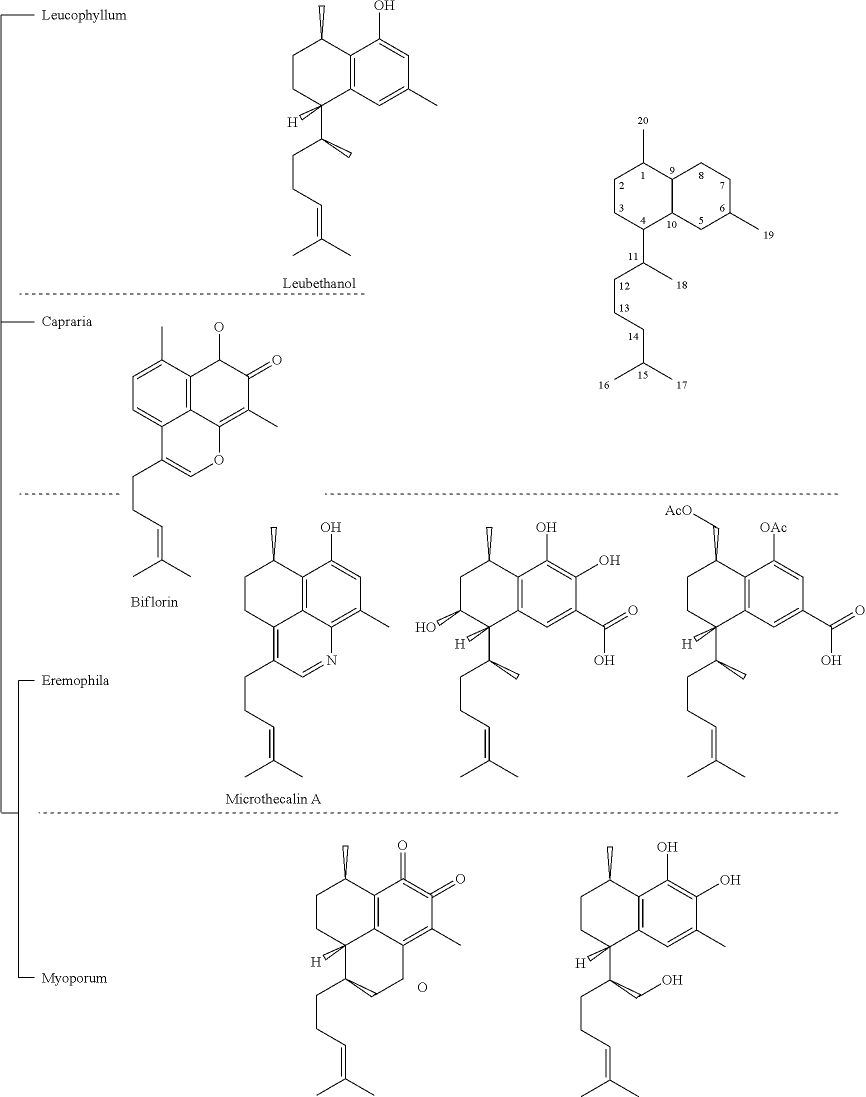

Fifteen candidates were identified, and a phylogenetic tree was constructed to group each candidate by TPS subfamily. One candidate (LfTPS13) was not expressed in root tissue and was eliminated from further consideration.

While containing a bicyclic decalin core, the structure of leubethanol is inconsistent with the labdane group of plant diterpenoids, the most common type of backbone which results from cyclization by pairs of class II and class I diTPS (Nat Prod Rep 27, 1521-1530 (2010)). In contrast, the cyclization pattern of leubethanol indicates activity of a class I enzyme, which catalyzes cyclization via removal of the diphosphate moiety. Out of the fourteen root-expressed candidates, only one was predicted to be a class II TPS (LfTPS4; TPS-c subfamily), and therefore thirteen possibilities remained.

A number of non-labdane diterpenes have been shown previously to be made by TPS-a enzymes which are localized to the plastid (PNAS 91, 8497-8501 (1994)). The majority of TPS-a enzymes are sesquiterpene synthases localized to the cytosol (The Plant Journal 66, 212-229 (2011)), and the presence of an N-terminal plastidial transit peptide in the primary amino acid sequence can therefore aid in prediction of diterpene synthase activity in this subfamily. Two L. frutescens candidates (LfTPS1 and LfTPS2) in the TPS-a subfamily were found to carry N-terminal extensions. Additionally, both have an ortholog in E. serrulata with nearly identical sequence length and homology through these N-terminal extensions. Of these two candidates, only LfTPS1 is exclusively expressed in root tissue and was therefore considered the more likely candidate, however both were tested.

Full-length genes for both LfTPS1 and LfTPS2 were cloned from root cDNA for transient expression in an N. benthamiana system engineered for increased levels of the presumed substrate GGPP. N-terminal truncated constructs, removing the putative transit peptides, were cloned into pET-28b(+) for expression of pseudomature variants in E. coli. Assays were extracted with hexane and analyzed by GC-MS.

To account for uncertainty of the predicted plastidial targeting signals, transient expression assays in N. benthamiana were carried out separately with co-expression of either plastidial or cytosolic GGPP terpene precursor pathway enzymes. Co-expression of both candidates with either cytosolic or plastidial precursor enzymes did not yield detectable products. To independently verify activity, each enzyme was expressed in E. coli with a C-terminal histidine tag and purified through Ni-affinity chromatography. Consistent with the results of the transient N. benthamiana assays, incubation of both LfTPS1 and LfTPS2 with GGPP in in vitro assays yielded no measurable activity. Additionally, no activity was seen when incubated with farnesyl diphosphate (FPP, precursor for sesquiterpenes) or geranyl diphosphate (GPP, precursor for monoterpenes).

### Example 3: LfTPS1 Exclusively Cyclizes Nerylneryl Diphosphate into the Serrulatane Backbone

Following these results, we considered two routes forward: first, to expand testing to each other class|TPS candidate, and second, to test LfTPS1 and LfTPS2 against uncommon terpene precursors. The former route was considered because even very closely related TPSs can have activities which differ substantially and there are many examples of TPSs which have different functions than would be predicted by their subfamily. The latter route was considered because of the absence of activity against each common substrate. GPP, FPP, and GGPP contain exclusively trans double bonds. All-cis stereoisomers of each have been reported in members of the nightshade (Solanaceae) family, together with TPSs which can convert these to terpene products.

The serrulatane backbone is ambiguous with respect to the original stereochemistry of its precursor; however, closer inspection of diterpenoids from the Eremophila genus shows that acyclic, bisabolane, and cembrane type diterpenoids in various Eremophila species contain internal cis double bonds.

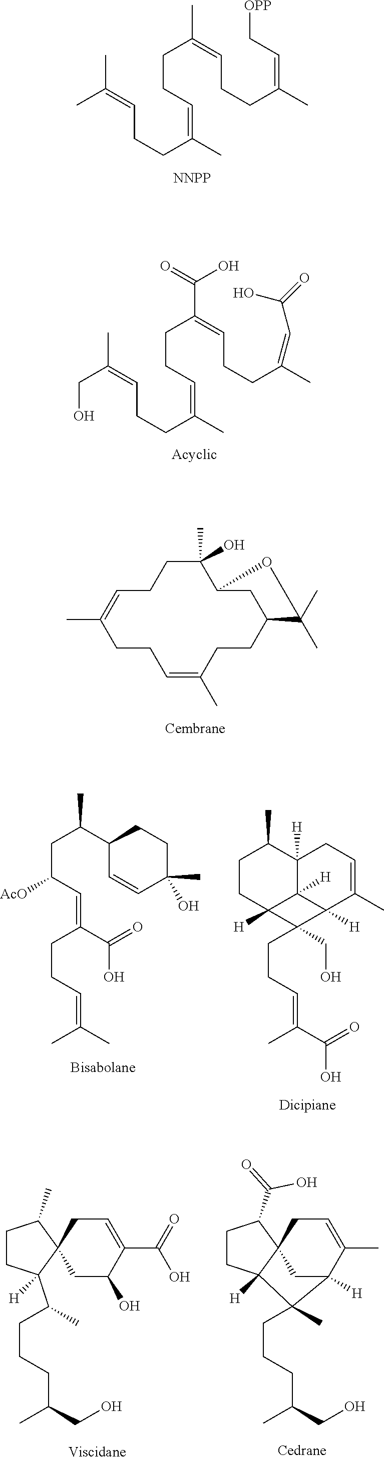

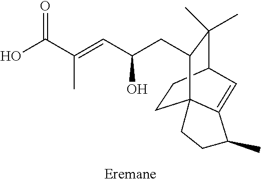 [SMILES: C/C(=C\[C@H](O)CC1C23CCC(C=C2[C@@H](C)CC3)C1(C)C)C(=O)O]

This prompted us to test NNPP (the all-cis stereoisomer of GGPP) as the precursor for the serrulatane backbone in L. frutescens. Since NNPP is not commercially available, truncated constructs of LfTPS1 and LfTPS2 in pET-28b(+) were used for co-expression with SICPT2, the plastidial S. lycopersicum cis-PT, in an E. coli system engineered to increase terpene precursor availability. Following hexane extraction and analysis by GC-MS, LfTPS1 was found to convert NNPP. This activity was independently confirmed in N. benthamiana. Four diterpene products were observed, with only one major product A:

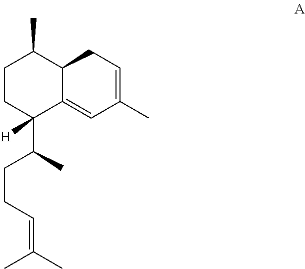 [SMILES: CC(C)=CCC[C@H](C)[C@@H]1CC[C@@H](C)[C@H]2CC=C(C)C=C12]

in the E. coli system, and a relative amount of another compound B exceeding:

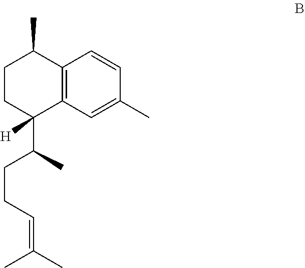 [SMILES: CC(C)=CCC[C@H](C)[C@@H]1CC[C@@H](C)c2ccc(C)cc21]

A in N. benthamiana. Diterpene olefins typically have a molecular ion of 272 m/z, however B has a molecular ion of 270 m/z. The fragmentation pattern for B is consistent with an aromatic product, and is similar to that of leubethanol (286 m/z) with major peaks shifted by 16, consistent with a difference of one hydroxylation. Given that TPSs are not known to catalyze redox reactions, B is likely derived from spontaneous aromatization of the major product A, a phenomenon seen previously in diterpene biosynthesis. To confirm the structure of A, production in the E. coli system was scaled up for NMR analysis, revealing that LfTPS1 makes dihydroserrulatene, and supporting the identity of B as serrulatene. In parallel to the testing against NNPP, we began working towards testing the remaining class I candidate TPSs. While we cloned each of these candidates out of L. frutescens cDNA, we received the positive results for LfCPT1 conversion of NNPP to dihydroserrulatene before we characterized these other candidates. These were, however, cloned and sequence verified, and are given here with GenBank accession numbers for reference.

### Example 3: LfCPT1, a Short Chain Cis-Prenyl Transferase, Supplies NNPP in Serrulatane Biosynthesis

We next sought out the source of NNPP in L. frutescens by searching for a cis-prenyl transferase. Cis-PTs are ubiquitous throughout plants and are typically involved in the synthesis of long chain polyisoprenoids (Akhtar et al., 2013), although very few which make short chain products (fewer than 35 carbons) have been identified. Three short-chain cis-PTs which yield NPP (neryl diphosphate; 10 carbon), (Z-Z)-FPP (Z-Z-farnesyl diphosphate; 15 carbon), and NNPP (20 carbon) have been identified from Solanum lycopersicum through functional characterization of the entire family of cis-PTs from this species. We identified candidate cis-PTs from both the L. frutescens and E. serrulata transcriptomes through a homology-based search against the entire family of cis-PTs from S. lycopersicum. Ten candidate cis-PTs were identified from L. frutescens, and phylogenetic analysis revealed that six are closely related to the short-chain cis-PTs from S. lycopersicum. LfTPS1 has a predicted plastidial transit peptide, and successfully converts NNPP in N. benthamiana assays when co-expressed with SICPT2, which is known to be targeted to the plastid. Therefore, we looked for a cis-PT candidate that is likely targeted to the plastid. Three of these candidates were found to carry predicted plastidial transit peptides and are expressed in root tissue (LfCPT1-3). LfCPT1 was considered to be the most likely candidate as it is the only of these three to have a direct ortholog in our E. serrulata transcriptome assembly (EsCPT1), however all three were tested.

LfCPT1-3 were cloned from L. frutescens root cDNA. Each candidate cis-PT was coexpressed in N. benthamiana with LfTPS1, and products were analyzed by GC-MS following hexane extraction. Co-expression with LfCPT1 yielded the same diterpene product profile as with the NNPP synthase from S. lycopersicum (SICPT2). In addition, direct comparison of LfCPT1 with SICPT2 without co-expression of a TPS showed the same peak and mass spectrum for dephosphorylated NNPP.

### Example 4: A Cytochrome P450 Converts the Serrulatane Backbone to Leubethanol

Leubethanol is oxidized twice relative to dihydroserrulatene, presumably through hydroxylation by a cytochrome P450 and aromatization. Given the propensity for dihydroserrulatene to spontaneously aromatize to serrulatene, we set out to identify P450 candidates for the required oxidation at C8. A homology-based search of both the L. frutescens and E. serrulata transcriptomes was carried out against a reference set of plant P450s. 165 candidates were identified from L. frutescens. We first narrowed our search by focusing on those within the CYP71 clan. While P450s in other clans have been identified in diterpenoid specialized metabolism, we began our search here based on the CYP71 clan containing the majority of previously characterized examples. Clustering each P450 candidate by family and eliminating those outside of the CYP71 clan reduced the list of candidates to 59. Considering only those that were expressed in root tissue but not flower tissue, and those that had an ortholog in our E. serrulata transcriptome assembly, only five candidates remained. One additional candidate (CYP71D615), which did not have a direct ortholog in E. serrulata, was included based on its root-exclusive expression and location among a cluster of other L. frutescens and E. serrulata candidates in the phylogenetic tree.

These six P450 candidates were cloned from L. frutescens root cDNA. Co-expression with LfCPT1 and LfTPS1 in N. benthamiana revealed that CYP71D616 facilitates the conversion of dihydroserrulatene to leubethanol. A relative decrease of dihydroserrulatene over serrulatene indicates that the preferred substrate for CYP71D616 is dihydroserrulatene. The observed minor reduction in serrulatene is plausibly due to P450-mediated turnover of dihydroserrulatene preceding spontaneous aromatization. This is supported by the metabolomic data from root tissue extracts, which shows an accumulation of serrulatene but no detectible quantities of dihydroserrulatene.

The interdependence of each enzyme in the pathway is demonstrated, showing that all three are necessary for leubethanol production when expressed in N. benthamiana. To determine whether the TPS activity is conserved in the Eremophila genus, we tested a synthetic homolog of LfTPS1 (EsTPS1; 85% amino acid identity) from the E. serrulata transcriptome assembly. Replacing LfTPS1 with EsTPS1 yields the same products in each combination, demonstrating orthology between the enzymes and conservation of this pathway in the serrulatane-rich Eremophila genus.

### Discussion

Through comparative transcriptomics between tissue types and genera, we have identified three enzymes responsible for the biosynthesis of the serrulatane diterpenoid leubethanol in L. frutescens. The stereochemistry at all three chiral centers in dihydroserrulatene matches that of every serrulatane diterpenoid identified from the Scrophulariaceae family wherever the stereocenter is retained in the final diterpenoid product. This, and the conserved function between LfTPS1 and EsTPS1, suggest that dihydroserrulatene is in fact the common precursor to all serrulatanes. Others have reported a similar pathway to dihydroserrulatene involving a cis-PT and plastidial TPS-a in Eremophila drummondii and Eremophila denticulata, further supporting the conservation of this pathway. BMC Plant Biology 20, 91 (2020). Nearly all of the serrulatane diterpenoids in Scrophulariaceae share a common hydroxylation (or derivative thereof) with leubethanol, suggesting that leubethanol itself is a common precursor. Given this commonality, the CYP71D616-catalyzed hydroxylation is likely the entry step between the diterpene backbone and diversification toward other antimicrobial serrulatane diterpenoids from other genera such as biflorin and microthecalin A.

This pathway is unusual in that it involves the all-cis prenyl diphosphate precursor NNPP rather than the common diterpene precursor GGPP. Prenyl diphosphate substrates are synthesized by members of either the trans-or cis-prenyl transferase families, typically in a head-to-tail condensation of the 5-carbon molecules isopentenyl diphosphate (IPP) and dimethylallyl diphosphate (DMAPP). These two enzyme families are distinct with no sequence or structural homology. The evolution of members of the cis-PT family to make uncommon terpene precursors has been found in two other cases, with the series of NPP (SICPT1), (Z,Z)-FPP (SICPT6), and NNPP (SICPT2) in S. lycopersicum (Solanaceae), and lavandulyl diphosphate (head-to-middle condensation catalyzed by LiLPPS) in Lavandula x intermedia (Lamiaceae). LfCPT1, LiLPPS, and the S. lycopersicum short-chain cis-PT are phylogenetically closely related when compared to the overall characterized cis-PT family in S. lycopersicum. This may indicate a shared common ancestry of the short-chain cis-PTs in Solanaceae, Lamiaceae, and Scrophulariaceae. Scrophulariaceae diverged from Solanaceae between 75 to 88 MYA, and from Lamiaceae between 44 and 67 MYA based on molecular time estimates, which is consistent with the divergence pattern of the short-chain cis-PTs: LfCPT1 appears to be more closely related to LiLPPS (Lamiaceae) than any of the Solanum cis-PTs, despite being closer to the Solanum enzymes in product profile. Additionally, it has been suggested that the shorter product length of the S. lycopersicum cis-PTs may be due in part to a shortened alpha helix not present in the long-chain cis-PTs from this species. This is not present in either LiLPPS or LfCPT1 based on homology modeling and a sequence alignment, suggesting that the evolution towards smaller precursors is independent and follows different trajectories from an ancestral sequence.

In addition to finding a similar pathway to dihydroserrulatene, others have identified TPSs which make the cembrane and viscidane backbones in Eremophila lucida, and showed that these exclusively use NNPP over GGPP as well. To identify where the TPSs and cis-PTs from these three other Eremophila species (E. denticulata, E. drummondii, and E. lucida) lie relative to our candidates, we generated phylogenetic trees including each candidate identified from these species and our sequences. Each other Eremophila NNPP synthase is a direct ortholog of LfCPT1, while LfCPT2 and LfCPT3 have no orthologs in any of these Eremophila species. Interestingly, a (Z,Z)-FPP synthase (EdCPT2) was found, however a TPS in Eremophila which converts (Z,Z)-FPP has yet to be identified. The cembratrienol synthase (ElTPS31) is a member of the TPS-b subfamily, commonly involved in monoterpene synthesis, and L. frutescens does not have an ortholog. The hydroxyviscidane synthase (ElTPS3) lines up closely with LfTPS2 and another enzyme from E. denticulata (EdfTPS5), however neither of these candidates were found to have this same function. Interestingly, more TPSa candidates which are putatively targeted to the plastid, but do not convert GGPP or NNPP, are present in these three Eremophila species. The function of LfTPS2 and these other plastidial TPS-a enzymes remains to be seen, and may suggest that other precursors that were not taken into account in either study may be present in the plastids of these plants.

The identification of a short-chain cis-PT in Scrophulariaceae clarifies the likely origin of other diterpene backbones present in the Eremophila genus. Acyclic and bisabolane type diterpenoids identified in this genus contain internal alkenes in cis configuration. As serrulatanes and viscidanes have now both been shown to be derived from NNPP, it is likely that the decipiane, cycloserrulatane, and cedrane backbones are derived from NNPP as well. The backbones for decipianes and cycloserrulatanes resemble a tricyclic serrulatane backbone, and the cedrane backbone resembles a tricyclic viscidane backbone. Beyond Scrophulariaceae, there are hundreds of other diterpene backbones with unknown biosynthetic routes. In Lamiaceae alone there are at least 200 (Johnson et al., 2019a), and in Salvia sclarea (Lamiaceae), two previously reported diterpenoids salviatriene A and B (Laville et al., 2012) resemble a cycloserrulatane and tricyclic viscidane, respectively. Given the independent emergence of cis-PTs which yield NNPP in different plant families, it may be that some of these unknown diterpenoid pathways involve NNPP as well.

Numerous diterpene backbones that differ from the more common labdane structure have been shown to be formed by enzymes in the TPS-a subfamily, which is mostly comprised of cytosolic sesquiterpene synthases. LfTPS1 provides another example of a compartment and substrateswitching TPS from the this subfamily, but differs from these previous examples in that it does not convert GGPP. In contrast to earlier work in P. vulgaris (Lamiaceae), where the enzyme PvHVS showed acceptance of both GGPP and the presumed non-native NNPP, LfTPS1 showed a high specificity towards NNPP. PvTPS5 and PvTPS2 (both TPS-a) could also convert NNPP to a diterpene product in addition to their native functions as sesquiterpene and diterpene synthases, respectively. This could plausibly arise from negative selection against GGPP, as both substrates are available in L. frutescens and presumably only GGPP is available in P. vulgaris. The presence of competing substrates in L. frutescens may introduce a strong selective pressure for specificity (Tawfik, 2014), while the absence of NNPP in P. vulgaris means that no such selective pressure exists. Such specificity can also be seen in Solanum where these all-cis substrates are present, where PHS1, SBS, and SlTPS21 all showed high specificity towards NPP, (Z,Z)-FPP, and NNPP, respectively compared to their all-trans counterparts.

Even some class II diTPSs (TPS-c) have been shown to have promiscuous activities in converting NNPP into irregular labdane structures. The substrate promiscuity of these TPSs suggests that the evolution of a prenyl transferase to afford an unusual terpene precursor may not require the co-evolution of a TPS, as the ability to convert a novel substrate may already be present in lineages where promiscuity was never selected against. Additionally, the occurrence of TPSs which natively convert cis-prenyl substrates is widespread throughout different TPS subfamilies. Examples have now been seen in the TPS e/f (Solanum species), TPS-b (Eremophila lucida), and TPS-a (L. frutescens and three Eremophila species) subfamilies, showing that evolution towards specificity for these substrates has happened independently in vastly different lineages of TPSs. Taken together, the presence of uncommon substrates may be more widespread than generally assumed, and the search for biosynthetic routes to new terpene backbones should involve a consideration of other possible precursors beyond the all-trans substrates which are typical.

The following statements are intended to describe and summarize various features of the invention according to the foregoing description provided in the specification and figures.

### Statements

An expression system comprising one or more expression cassettes, each expression cassette comprising a promoter operably linked to a nucleic acid segment encoding at least one of the following enzymes: a cis-prenyl transferase, a terpene synthase, a cytochrome P450, or a combination thereof.

The expression system of statement 1, wherein the cis-prenyl transferase, the terpene synthase, or the cytochrome P450 nucleic acid segment is from a Leucophyllum frutescens (Lf), Tripterygium wilfordii (Tw), Euphorbia peplus (Ep), Coleus forskohlii (Cf), Ajuga reptans (Ar), Perovskia atriciplifolia (Pa), Nepeta mussini (Nm), Origanum majorana (Om), Hyptis suaveolens (Hs), Grindelia robusta (Gr), Leonotis leonurus (Ll), Marrubium vulgare (Mv), Vitex agnus-castus (Vac), Euphorbia peplus (Ep), Ricinus communis (Rc), Daphne genkwa (Dg), or Zea mays (Zm) organism.

The expression system of statement 1 or 2, wherein the cis-prenyl transferase, the terpene synthase, or the cytochrome P450 enzyme is from a Leucophyllum frutescens (Lf).

The expression system of statement 1, 2 or 3, further comprising one or more expression cassettes, each expression cassette comprising a promoter operably linked to a nucleic acid segment encoding at least one of the following enzymes: more transcription factor, terpene synthase, cytochrome P450 reductase, 1-deoxy-D-xylulose 5-phosphate synthase (DXS), 1-deoxy-D-xylulose 5-phosphate-reducto-isomerase, cytidine 5′-diphosphate-methylerythritol (CDP-ME) synthetase (IspD), 2-C-methyl-d-erythritol 2,4-cyclodiphosphate synthase (IspF), geranylgeranyl diphosphate synthase (GGDPS), HMG-COA synthase, HMG-CoA reductase (HMGR), mevalonic acid kinase (MVK), phosphomevalonate kinase (PMK), mevalonate-5-diphosphate decarboxylase (MPD), isopentenyl diphosphate isomerase (IDI), abietadiene synthase (ABS), farnesylpyrophosphate synthase (FPPS), ribulose bisphosphate carboxylase, squalene synthase (SQS), patchoulol synthase, or WRI1 protein.

A host cell comprising an expression system comprising one or more expression cassettes, each expression cassette comprising a promoter operably linked to a nucleic acid segment encoding at least one of the following enzymes: a cis-prenyl transferase, a terpene synthase, a cytochrome P450, or a combination thereof.

The host cell of statement 5, wherein the cis-prenyl transferase, the terpene synthase, or the cytochrome P450 nucleic acid segment is from a Leucophyllum frutescens (Lf), Tripterygium wilfordii (Tw), Euphorbia peplus (Ep), Coleus forskohlii (Cf), Ajuga reptans (Ar), Perovskia atriciplifolia (Pa), Nepeta mussini (Nm), Origanum majorana (Om), Hyptis suaveolens (Hs), Grindelia robusta (Gr), Leonotis leonurus (Ll), Marrubium vulgare (Mv), Vitex agnus-castus (Vac), Euphorbia peplus (Ep), Ricinus communis (Rc), Daphne genkwa (Dg), or Zea mays (Zm) organism.

The host cell of statement 5 or 6, wherein the cis-prenyl transferase, the terpene synthase, or the cytochrome P450 enzyme is from a Leucophyllum frutescens (Lf).

The host cell of statement 5, 6 or 7, further comprising one or more expression cassettes, each expression cassette comprising a promoter operably linked to a nucleic acid segment encoding at least one of the following enzymes: more transcription factor, terpene synthase, cytochrome P450 reductase, 1-deoxy-D-xylulose 5-phosphate synthase (DXS), 1-deoxy-D-xylulose 5-phosphate-reducto-isomerase, cytidine 5′-diphosphate-methylerythritol (CDP-ME) synthetase (IspD), 2-C-methyl-d-erythritol 2,4-cyclodiphosphate synthase (IspF), geranylgeranyl diphosphate synthase (GGDPS), HMG-COA synthase, HMG-COA reductase (HMGR), mevalonic acid kinase (MVK), phosphomevalonate kinase (PMK), mevalonate-5-diphosphate decarboxylase (MPD), isopentenyl diphosphate isomerase (IDI), abietadiene synthase (ABS), farnesylpyrophosphate synthase (FPPS), ribulose bisphosphate carboxylase, squalene synthase (SQS), patchoulol synthase, or WRI1 protein.

A method comprising contacting terpene or terpenoid substrate with one or more of the following enzymes cis-prenyl transferase, a terpene synthase, a cytochrome P450 to thereby synthesize at least one serrulatane.

The method of statement 9, wherein the product comprises leubethanol (1).

The method of statement 9 or 10, which is performed in vitro in a cell-free mixture.

The method of statement 9, 10 or 11, which is performed within a cell that expresses at least one of the enzymes.

The method of statement 12, wherein the cell is a host cell comprising an expression system comprising one or more expression cassettes, each expression cassette comprising a promoter operably linked to a nucleic acid segment encoding at least one of the following enzymes: a cis-prenyl transferase, a terpene synthase, a cytochrome P450, or a combination thereof.

The specific methods, expression systems, and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention.

The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

One of ordinary skill in the art will recognize that the methods of the current disclosure can be achieved by administration of a composition described herein comprising at least one bronchodilator and at least one pulmonary surfactant via devices not described herein.

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading can occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

In the methods described herein, the steps can be carried out in any order without departing from the principles of the invention, except when a temporal or operational sequence is explicitly recited. Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more.

The term “substantially no” as used herein refers to less than about 30%, 25%, 20%, 15%, 10%, 5%, 3%, 2%, 1%, 0.5%, 0.1%, 0.05%, 0.001%, or at less than about 0.0005% or less or about 0% or 0%.

Those skilled in the art will appreciate that many modifications to the embodiments described herein are possible without departing from the spirit and scope of the present disclosure. Thus, the description is not intended and should not be construed to be limited to the examples given but should be granted the full breadth of protection afforded by the appended claims and equivalents thereto. In addition, it is possible to use some of the features of the present disclosure without the corresponding use of other features. Accordingly, the foregoing description of or illustrative embodiments is provided for the purpose of illustrating the principles of the present disclosure and not in limitation thereof and can include modification thereto and permutations thereof.

What is claimed is:

1. A method of making at least one serrulatane comprising contacting a terpene or a terpenoid substrate with at least one of a cis-prenyl transferase, a terpene synthase, and a cytochrome P450.

2. The method of claim 1, wherein the product comprises leubethanol (1).

3. The method of claim 1, which is performed in vitro in a cell-free mixture.

4. The method of claim 1, which is performed within a cell that expresses at least one of the enzymes.

5. The method of claim 1, wherein the cell is a host cell comprising an expression system comprising one or more expression cassettes, each expression cassette comprising a promoter operably linked to a nucleic acid segment encoding at least one of a cis-prenyl transferase, a terpene synthase, and a cytochrome P450.

6. The method of claim 1, wherein the serruletane is a compound of the formula: 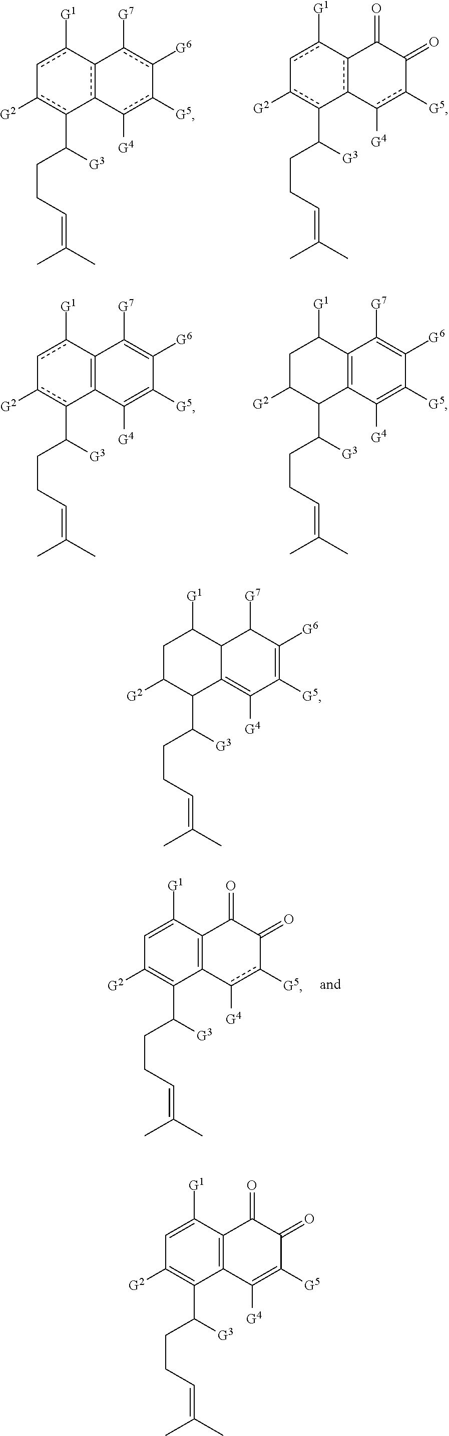
wherein:
G¹ is substituted or unsubstituted alkyl;
G² is H, substituted or unsubstituted alkyl or OG⁸, wherein G⁸ is H, substituted or unsubstituted alkyl or acyl;
G³ is substituted or unsubstituted alkyl;
G⁴ is H, substituted or unsubstituted alkyl, or G⁴ and G³, together with the atoms to which they are attached, can form a five- or six-membered heterocyclyl;
G⁵ is substituted or unsubstituted alkyl or C(O)OG⁸; and
G⁶ and G⁷ are each, independently, H or OG⁸.

7. The method of claim 1, wherein the serruletane is a compound of the formula:

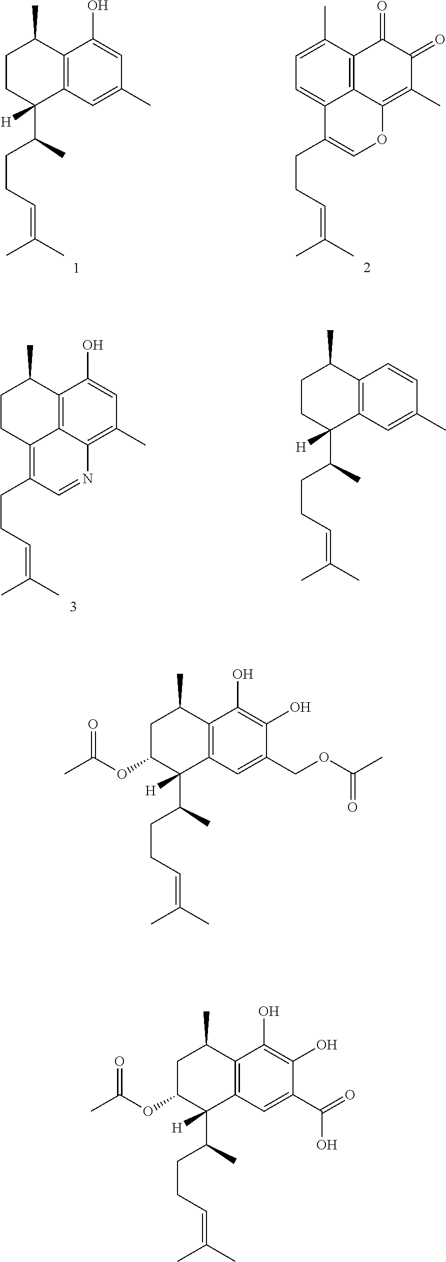

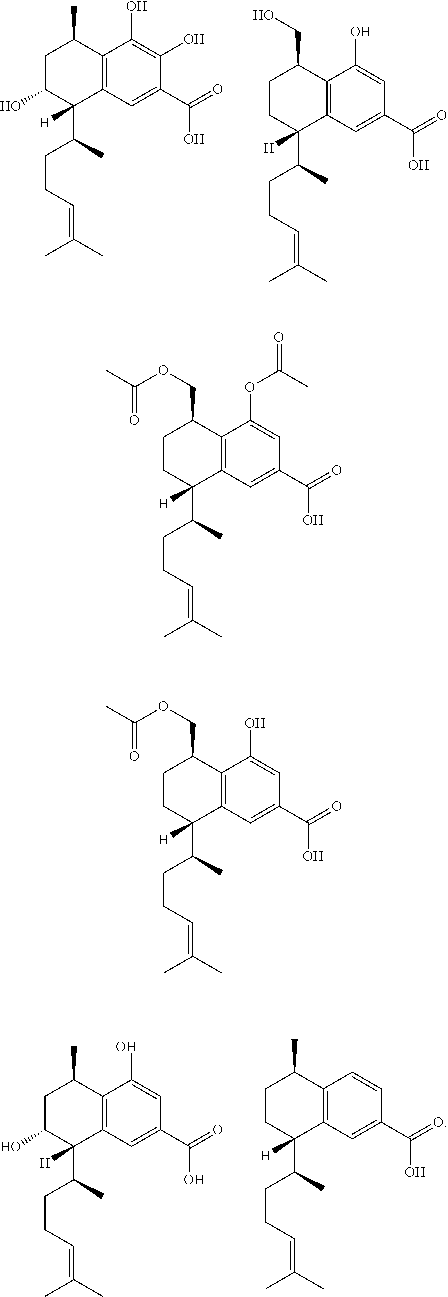 [SMILES: CC(C)=CCC[C@H](C)[C@H]1c2cc(C(=O)O)c(O)c(O)c2[C@H](C)C[C@H]1O ; CC(C)=CCC[C@H](C)[C@@H]1CC[C@@H](CO)c2c(O)cc(C(=O)O)cc21 ; CC(=O)OC[C@@H]1CC[C@@H]([C@@H](C)CCC=C(C)C)c2cc(C(=O)O)cc(OC(C)=O)c21 ; CC(=O)OC[C@@H]1CC[C@@H]([C@@H](C)CCC=C(C)C)c2cc(C(=O)O)cc(O)c21 ; CC(C)=CCC[C@H](C)[C@H]1c2cc(C(=O)O)cc(O)c2[C@H](C)C[C@H]1O ; CC(C)=CCC[C@H](C)[C@@H]1CC[C@@H](C)c2ccc(C(=O)O)cc21]

8. The method of claim 1, wherein the terpene or terpenoid substrate is a compound of the formula: 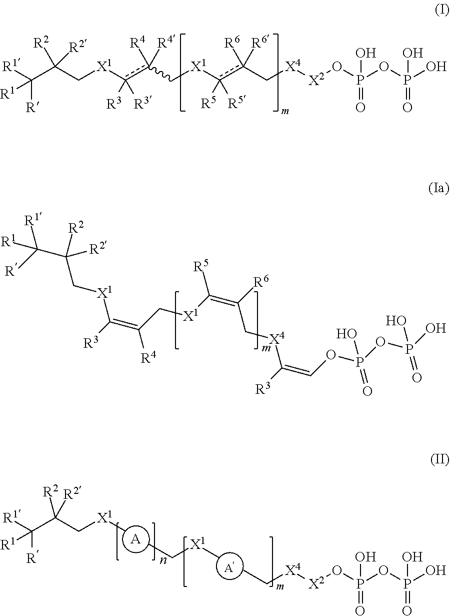
wherein:
m is an integer from 0 to 3, with the understanding that if m is 2 or 3, each repeating subunit can be the same or different;
n is an integer from 0 to 1;
the dashed lines (-----) represent a double bond when R^{3′} and R^{4′} are absent or when R^{5′} and R^{6′} are absent,
A and A′ are each independently cycloalkyl, aryl or heterocyclyl, each of which can be optionally substituted;
X¹ is a heteroatom, -X³-alkyl, -alkyl-X³- or alkyl, wherein X³ is a heteroatom or alkyl or X¹ is: 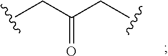
R¹ and R² form a double bond or an epoxide;
each R′, R^{1′}, R², R^{2′}, and R³-R⁶ is, independently, H, alkyl, halo, aryl, and alkylaryl;
R^{3′} and R^{4′} are absent or R^{3′} and R^{4′}, together with the carbon atoms to which they are attached, form an epoxide, a cycloalkyl group, an aryl group or a heterocyclyl group;
R^{5′} and R^{6′} are absent or R^{5′} and R^{6′}, together with the carbon atoms to which they are attached, form an epoxide, a cycloalkyl group, an aryl group or a heterocyclyl group;
X² is a bond, alkenyl or acyl; and
X⁴ is a absent, a heteroatom or alkyl;
with the proviso that the compound of the formula (I) is not a compound of the formula:

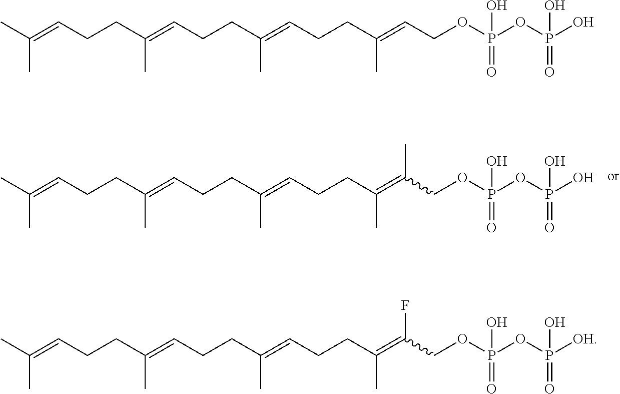 [SMILES: CC(C)=CCC/C(C)=C/CC/C(C)=C/CC/C(C)=C/COP(=O)(O)OP(=O)(O)O ; CC(C)=CCC/C(C)=C/CC/C(C)=C/CCC(C)=C(C)COP(=O)(O)OP(=O)(O)O ; CC(C)=CCC/C(C)=C/CC/C(C)=C/CCC(C)=C(F)COP(=O)(O)OP(=O)(O)O]

9. The method of claim 1, wherein X¹ is a heteroatom and the heteroatom is oxygen.

10. The method of claim 1, wherein X³ is oxygen or C₁-C₅-alkyl.

11. The method of claim 1, wherein R³-R⁶ are each H or C₁-C₅-alkyl.

12. The method of claim 1, wherein R³ and R⁵ are each H or C₁-C₅-alkyl; and R⁴ and R⁶ are each H.

13. The method of claim 1, wherein m is 1 or 2.

14. The method of claim 1, wherein m is 0.

15. The method of claim 1, wherein X² is an alkenyl group of the formula: 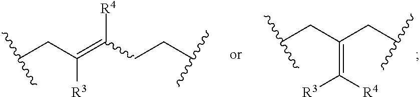
or an acyl group of the formula:

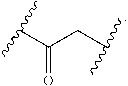 [SMILES: *CC(*)=O]

16. The method of claim 1, wherein the terpene or terpenoid substrate is a compound of the formula: 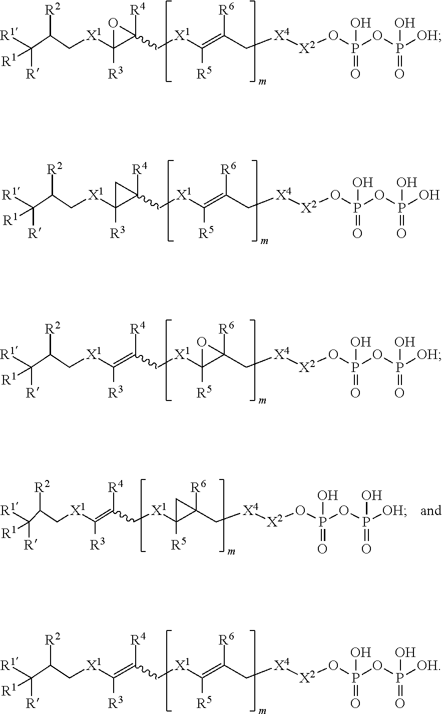
wherein:
m is an integer from 0 to 3, with the understanding that if m is 2 or 3, each repeating subunit can be the same or different;
X¹ is a heteroatom, -X³-alkyl, -alkyl-X³- or alkyl, wherein X³ is a heteroatom or alkyl or X¹ is: 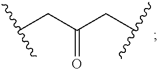 [SMILES: *CC(=O)C*]
R¹ and R² form a double bond or an epoxide;
each R′, R¹, R², and R³-R⁶ is, independently, H, alkyl, halo, aryl, and alkylaryl;
X² is a bond, alkenyl or acyl; and
X⁴ is a absent, a heteroatom or alkyl.

17. The method of claim 1, wherein the terpene or terpenoid substrate is a compound of the formula: 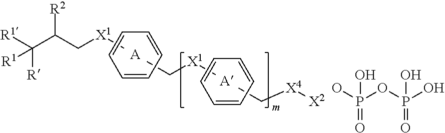
wherein:
m is an integer from 0 to 3, with the understanding that if m is 2 or 3, each repeating subunit can be the same or different;
A and A′ are each independently cycloalkyl, aryl or heterocyclyl, each of which can be optionally substituted;
X¹ is a heteroatom, -X³-alkyl, -alkyl-X³- or alkyl, wherein X³ is a heteroatom or alkyl or X¹ is: 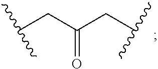 [SMILES: *CC(=O)C*]
R¹ and R² form a double bond or an epoxide;
each R′, R¹, R², and R³-R⁶ is, independently, H, alkyl, halo, aryl, and alkylaryl;
X² is a bond, alkenyl or acyl; and
X⁴ is a absent, a heteroatom or alkyl.

18. The method of claim 1, wherein the terpene or terpenoid substrate is a compound of the formula: [chemical structure]


SEQUENCE LISTING (6 sequences)

SEQ ID NO: 1 - protein, 329 aa, Leucophyllum frutescens
MQISLQFPKASPCTIKSFTTPSPPPHHGDVETPCCVSSIKIGTPWKKTMVDHGLSFARFSPAAPLSSKFRLAARVEDREISGEIQLPDALQAELMPKHVAVIMDGHGRWAENRGLPIQHGHNAGLENLKQLLLHCCKFGIGVLSVYAFSTENWKRTKEEIDFLMSGYECFIQYYVKDLILRHDLQFSVIGDKSRLPQSIRSTIASAEEAGKANSGTHFVMALSYSGQYDIVDASKKIASQVGSGKLRAEDIDESVFEQQLLTNVTKFPNPDLLIRTSGELRVSNFMLWQLAYAEFYFVDKLFPDFEEADFIEALSSFQQRKRRYGGRKK

SEQ ID NO: 2 - DNA, 990 bp, Leucophyllum frutescens
  source 1..990: mol_type = genomic DNA
atgcagatctcccttcagttcccaaaagcttccccatgcactatcaaaagtttcaccactcctagtcctcctcctcatcacggggacgtcgaaacaccctgctgcgtatcttccatcaaaataggcaccccttggaagaaaacaatggtggatcatggactgagctttgcacgtttttctccagctgcacctttgtcctctaagttccgtttggcagctcgagtcgaagaccgtgaaattagtggtgagatccagttgccggacgctctgcaggctgaactgatgcctaaacacgtcgctgtgatcatggatggccacggtaggtgggccgagaacagagggctgcccatacaacacggccacaacgcaggtctggagaacttgaaacaactgcttctgcactgctgcaaatttggaattggggttctttcggtgtatgccttctccacggagaactggaagcgcactaaggaagaaatcgacttcttgatgagcggttatgagtgtttcatacaatattacgtgaaggatctaatactgagacatgatctgcaattttctgttattggagataaatctaggcttcctcagtctatacgatctacaatagcttcggctgaggaagccgggaaggccaacagtgggacacattttgtgatggcgctgagctatagcggtcaatacgacatagtagatgcaagcaagaaaattgcaagccaagtgggaagcgggaaactacgtgcagaagatatagatgaaagtgtgttcgagcaacaactattgacgaacgtcacaaagtttccgaaccccgacttgctcatacgaacaagcggagaattaagagtcagcaatttcatgttatggcaactggcctacgcagagttctacttcgtcgacaaactgttcccagattttgaagaggccgactttatagaggccctgagttcattccaacagaggaaaaggcgttatggtggacgaaagaaatga

SEQ ID NO: 3 - protein, 608 aa, Leucophyllum frutescens
MAAPTGFAQCGTKDIFRLPVNVNGRRSFLYGIQTKSQYSKYQKHNHGGLCLRPMAAATLDLDGQEDTVSSSSVKGHHRPSSWRTISFSFDNQVQERYAEAVEALKEEVRAMVMAKDSKPREKMNLIDTLERLGVAYHFKHEIEEQIEQIFKSHAKDDGPDSDLFTTALYFRLCRQHGYDVNSSMFGRFKGKDGKFEKGLISDINGLLSLYEASYLRYHGEDILEEATVFTTHYLNEAKPQILDPYLREKVARALKQPLHRGVEKLESRYYISVYEKYESRNELLLKLAKLDFNILQNLYKKELSELFKWWKELDLTSKLPYVRDRVAECFFWGMAMSYEPEYSLSRVAAAKAIVMITVLNDTYENVSTLKQLEIFPEIVQRWDTKDNDQLPAYMKIAYEFLMGVYEDHDSNVSKQGRSYAVPYAIETMIQLAKAYHKKAKWYTGEEMPTFEDHVSNGAVLSTIYVLLSSFYIGTESASEEAFVWLINRPTIVDAVGLLGRYVNDIGTYERECKGGQLSTTAIDCYTRENGVSREETLNKFLEFAEDTWMTINKEWVTASCVPRDIMRPVLNLGRVGDTTYKGGTDGYTDPESGLEQDIFALFLKPIDI

SEQ ID NO: 4 - DNA, 1827 bp, Leucophyllum frutescens
  source 1..1827: mol_type = genomic DNA
atggctgctcccaccggctttgctcaatgtggtacaaaagatattttccggctgcctgtgaacgttaatggtcgccggagctttctatatggtatccaaacaaaatctcagtactctaaatatcagaaacacaatcatggaggcttgtgtttgagacccatggctgcagccacccttgatcttgatggtcaagaagacaccgtcagttccagttctgtgaagggtcatcatcgcccgagctcgtggagaactatctcattctcttttgacaatcaggtacaagaaagatacgcagaagctgttgaagcattaaaggaagaagtaagggccatggtgatggccaaagacagtaaaccaagagagaaaatgaatttgattgacacgcttgaacgcttgggagtcgcttatcactttaagcatgaaattgaagagcagatcgaacagatcttcaagtctcatgcaaaagacgatggacctgattctgatcttttcactactgcactctattttcgtctgtgtagacaacatggatatgacgtcaattccagtatgtttggtcggttcaagggcaaggatgggaagttcgaaaagggtcttattagtgacattaacggtttactgagcctgtacgaagcaagttatttgaggtatcatggggaggacatcttagaagaagccaccgtcttcacgacacattacttaaatgaggcaaaaccacaaatattagacccttatcttcgagaaaaagtggcaagagccctaaagcaaccgcttcacaggggcgttgagaaattggagtcacgttattacatttcagtatacgagaaatatgagtcgaggaatgagctgcttctgaaactagccaaactggatttcaacatattgcaaaacctgtacaaaaaggagctcagtgagctgttcaagtggtggaaggagttggatcttacatcaaaacttccatatgtgagggatagagtggcggaatgctttttttggggcatggcgatgagctatgaacccgagtattccttgtctcgagtggctgctgccaaggccatagtaatgatcaccgttctgaatgatacgtatgaaaacgtttcaacccttaaacaactggaaatctttcctgagattgtgcaaagatgggatactaaagacaatgatcagctcccagcttatatgaagattgcttatgaatttctgatgggtgtatacgaggaccacgatagcaacgtgtcaaaacagggaagatcttatgctgtgccctatgccatagaaacgatgatacaacttgcaaaggcttaccacaagaaggcgaaatggtacactggagaagagatgccgacatttgaagaccacgtttccaatggggctgtcctgagtaccatctacgtgttgctgtcgtcattttacatcggcacagagtccgcctccgaagaagcgtttgtttggctcataaatcggcctacaattgtggatgctgttggtcttctcggtcgatacgtgaatgatatcggcacctacgaacgtgagtgcaagggaggccagctttctactacggcaattgattgctacacgagagagaatggtgtttcgagagaagaaactctgaataaattccttgaatttgcggaggatacgtggatgacaatcaacaaggaatgggttactgcaagctgtgtacccagagatattatgaggccagttcttaaccttggccgcgtcggagatactacctataagggcggtacggatgggtataccgatccagagagcggcctcgaacaagacatttttgctctatttctaaagccgatcgacatttag

SEQ ID NO: 5 - protein, 599 aa, Leucophyllum frutescens
MASGITGIQYGSIEILHQPGRRNFLSDISSSIQNQLKKKYQYELTNHNKQLKYRGLCLSRPMAAAEVLDDQEDNTTLMTSVKAHDSNFLGAISFSFGNQAPFKFTTSLLIFSSFMFLLVKSSEKTKVPARKYENLPPSPPKLPLIGHLHHLLGGLPHHTLARVTEKFGPVVHLQIGEISTVVISSPEAAKEVLKVRDTACANRPQSISIEIMLYNYADFVFAPYDEFWRQMRKICIMEMLSARNVKSYGSIRQDEVLHLIKSLQSASGRAINLSEKIFATTSSIVCRVAFGKVLRDRDTLIDLMKKGISLAAGFELVDVFPSFKMLHAVSWNRNKLLKMHKELDAILDTIVEGHKLKENGEYGGEDIVDVLLRMKESGELKFPITNENIKAVIFDVFAAATDTSSSTVDWAMAELVKNPNAYAKAQAEVRQAFTREEIVDAERHLHKLNYLKLVIKETLRLHPPVPLLPRASREECEVNGYSIPLNSKVLVNIWSMGRDPKYWDEPESFRPERFENNNIEFFGNNFEYIPFGSGKRICPGISFGMANVELPLAHLLYHFDWKLPEGMTTADVDLTEAYGLAVIRKNALVVEPTSYNPST

SEQ ID NO: 6 - DNA, 1800 bp, Leucophyllum frutescens
  source 1..1800: mol_type = genomic DNA
atggcttctgggatcaccggtatccaatatggttcgatagagatcctccatcagcccggccgccggaactttctgagtgatatttcgtcctctatacagaatcagcttaagaagaagtatcagtatgaactgaccaatcataataagcaactcaagtacagaggcttatgcttaagtagacccatggctgccgccgaggtgcttgatgatcaagaagacaatactactctcatgacttctgtcaaggctcacgactccaacttccttggtgccatttcattttcttttggcaatcaggcacctttcaagttcaccaccagtctcctgattttttcatctttcatgttcctactggtcaagtcatcggagaaaaccaaagtacccgcaagaaaatacgaaaacttgcccccaagcccaccaaagctgcctctgataggccacctccaccacttgttgggtgggctgccacaccatacgctagccagagtaaccgaaaagttcggcccggtggtgcatcttcagataggagaaatttctacggtggtgatttcgtcaccggaggcagccaaagaggtgctgaaagttcgcgatactgcctgcgcaaataggcctcaaagtatcagcattgagatcatgttatacaattacgcggactttgtctttgctccttacgacgagttctggaggcagatgcgtaagatatgcatcatggagatgctcagcgctaggaatgtcaagtcttatggatctatcaggcaagatgaggtgttacatctcatcaaatctttgcagtcagcatctggaagagccatcaatctgagcgagaagatatttgcgacgacgagttccattgtgtgtagggtggcattcgggaaggtgttgagggatagggacacacttatagatctaatgaaaaagggaatttccctggcagcggggtttgagctggttgatgtgtttccatccttcaagatgttgcacgctgtgagttggaatagaaacaagttgttgaagatgcacaaggagcttgacgccattcttgataccattgttgaaggacacaagttgaaggagaatggtgaatatggaggtgaagatattgtagacgttcttctcagaatgaaggaaagtggagaactcaaatttccgattactaacgaaaacatcaaagctgttatttttgacgtattcgcagctgcgacagatacctcgtctagtaccgtagattgggccatggcagaacttgtgaaaaatcctaacgcgtacgcaaaggcacaagctgaagtaagacaagcatttacaagagaggaaattgtggatgcggaacgccacctccataagttgaactatctcaaacttgtgatcaaagaaactctcagattgcaccctccagtcccattactccccagagcaagcagggaagaatgtgaagtgaatggctattccatccctctcaattctaaagtgttggtcaatatttggtccatgggaagggatcctaaatactgggatgaacccgaaagctttcgacccgagaggtttgagaataataacatagaattctttgggaacaactttgaatacatcccattcggatcaggaaaaaggatatgtcccggtataagtttcggaatggcgaatgtggagctcccactggctcacctcctctatcatttcgactggaaattaccagagggaatgacaactgctgatgtagacttgactgaggcgtatggacttgctgtcataagaaagaatgctcttgtcgtcgaacccacatcttacaatccttccacttag